(19) United States
(12) Patent Application Publication
(10) Pub. No.: US 2025/0005759 A1
(43) Pub. Date: Jan. 2, 2025

(54) DEVICE AND METHODS FOR MONITORING HEART FUNCTION FOR TREATMENT OF CONGESTIVE HEART FAILURE AND OTHER CONDITIONS

(71) Applicant: BioVentrix, Inc., Mansfield, MA (US)

(72) Inventors: Paulo NEVES, Vila Nova De Gaia (PT); Sean MCMANUS, Dallas, TX (US)

(21) Appl. No.: 18/701,197

(22) PCT Filed: Oct. 14, 2022
(86) PCT No.: PCT/US2022/046659

§ 371 Date: Apr. 12, 2024

Related U.S. Application Data
(60) Provisional application No. 63/256,371, filed on Oct. 15, 2021.
(60) Provisional application No. 63/288,090, filed on Dec. 10, 2021.

(51) Int. Cl. G06T 7/00; A61B 5/11; G06T 7/246; G16H 15/00; G16H 40/63

(52) U.S. Cl. CPC G06T 7/0016; A61B 5/1107; A61B 5/1128; G06T 7/248; G16H 15/00; G16H 40/63; G06T 2207/30048; G06T 2207/30204

(57) ABSTRACT

Techniques for assessing the contractility of various regions of the heart wall, including for example, the left and right ventricle of the heart are provided. An inward displacement value may be calculated that represents how much, or how little, the heart wall moves during contraction and relaxation. Assessment using the inward displacement value can be used to determine details specific to surgical implantations, including anchors. The assessment can further determine qualified candidates to undergo various surgeries and medical interventions.

### CROSS REFERENCE(S) TO RELATED APPLICATION(S)

This application claims priority to U.S. Provisional Application No. 63/288,090, filed Dec. 10, 2021, and U.S. Provisional Application No. 63/256,371, filed Oct. 15, 2021, the entire contents of each being hereby incorporated by reference.

### TECHNICAL OVERVIEW

The technology described herein relates to techniques for detection and treatment of heart failure. More particularly, the technology described herein relates to measuring inward displacement of the heart at, or along, one or more heart segments.

### INTRODUCTION

Quantifiable measurements of heart function are important to monitor and detect heart complications and potentials for heart failures. In particular, imaging processes are used for left ventricle (LV) and right ventricle (RV) function assessment. The most desirable of these means to monitor the heart involve procedures which are non-intrusive into the body, to allow for ease of monitoring without surgery. Currently, many physicians utilize echocardiography, Cardiovascular Magnetic Resonance (CMR) or Computed Tomography (CT) views.

From echocardiography, CMR or CT, heart function can be quantitatively recorded using Ejection Fraction (EF). Despite some drawbacks, EF is a good means of assessing overall LV and RV function of a patient's heart. However, EF does not provide information about the circulation. A low EF may be due to low stroke volume or increased LV diastolic volume. LV EF is mainly affected by preload, afterload, and contractility and absolute LV volumes reflect these factors differently. LV dysfunction is better defined using 2D echocardiography that can depict thinning and regional wall motion. Additionally, EF only assesses global LV and RV function, providing no information about regional (segmental) contractility.

More recently, another method which has emerged to quantify LV and RV function is global strain. This measure has been able to detect early ventricular dysfunction in subjects with normal ejection fraction. Strain analyzes myocardial deformation using speckle-tracking (echocardiography) or feature-tracking technology (CMR and CT). Global longitudinal strain, using 3 long-axis views of the LV, is the strain parameter more commonly used.

Strain can also be used for segmental LV analysis, as it can analyze the amount of myocardial deformation segment by segment. However, there are several drawbacks in this analysis, such as those associated with the interference of deformation (strain) on a given segment caused by adjacent segments. When applied to dilated hearts with akinetic and dyskinetic segments, this method could not determine which areas were akinetic or dyskinetic.

While valuable, echocardiography can be operator dependent, and suboptimal images may be acquired, making its post-processing prone to errors, mainly in severely dilated hearts, where it is easy to foreshorten left ventricle apex from the apical views.

Accordingly, it will be appreciated that new and improved techniques, systems, and processes are continually sought after.

### SUMMARY

As discussed herein, function and health of a patient's heart using a new approach can be measured using inward displacement of the heart at, or along, one or more heart segments. This method assesses the regional contractility of various regions of the heart wall motion, including for example, the left and right ventricle. Such assessment can provide information to determine details specific to surgical implantations, including anchors. The assessment can further determine qualified candidates to undergo various surgeries and medical interventions. Both applications are discussed further below.

This method and associated devices can be used for detection and treatment of various pathologies. For example, inward displacement measurement methods may be used for monitoring congestive heart failure, myocarditis, amyloidosis, and heart rhythm disturbances. In some implementations, inward displacement may be used to monitor high-risk or damaged hearts or damaged aspects of a heart. In other implementations, inward displacement may provide a means of detecting failure in hearts, or other conditions, that have no history of disease of heart condition and which are difficult to detect using conventional means.

In a first embodiment, a computer-implemented method for monitoring heart function for treatment of congestive heart failure and other conditions is provided. The method comprises obtaining a plurality of images of a patient's heart or a portion thereof and obtaining an inward displacement measurement of at least a region or segment of the heart based on the difference in the heart wall position between different images of the plurality of images. The method comprises assessing the regional contractility of the heart wall motion based on the inward displacement measurement; and providing a graphical representation of the regional contractility of the heart wall for determining regions or segments of the heart that are suitable or eligible for therapeutic treatment.

Advantageously the method of the first embodiment may be used for monitoring congestive heart failure, myocarditis, amyloidosis, and heart rhythm disturbances. Advantageously the method of the first embodiment may be used to monitor high-risk or damaged hearts or damaged aspects of a heart. For example, the method of the first embodiment may be used as a means of detecting failure in hearts, or other conditions, that have no history of disease or heart conditions, and which are difficult to detect using conventional means. Inward displacement may allow direct comparison between regions or segments and an objective determination of which regions or segments are akinetic or dyskinetic, which may aid in determining areas of the heart that are suitable or eligible for therapeutic treatment, such as ventricular reconstruction by means of anchor deployment, injection of hydrogels within the myocardium, stem cell therapies within the damaged myocardium, etc. Inward displacement may also increase the objectiveness of scar determination, mainly in CT, where late gadolinium enhancement is not available. The identification of scar tissue may aid in determining a proper placement of heart anchors for use in reconstructing the ventricle. Inward displacement may accurately represent by a quantifiable number the contractility of a given region or segment, making possible intra and inter-heart comparisons.

In a second embodiment, of the first embodiment, a method is provided that comprises obtaining a plurality of images of a patient's heart or a portion thereof comprises obtaining a series of cuts of the heart, wherein obtaining an inward displacement measurement of at least one region of the heart based on the difference between the heart wall position comprises, for each of the cuts of the heart. The method comprises determining a distance from a centerline to the end diastole and the centerline to end systole; and assigning a displacement by subtracting the distance from the centerline to the end diastole and the distance from the centerline to end systole.

Advantageously by using the method of the second embodiment, the series of cuts may be used to create a three-dimensional representation of the movement of the heart. A positive number between the end diastole and end systole may mean that the region of the heart is contracting inwards. A number which is zero, or approximately zero, may mean, or be associated with, akinesia. A negative number may mean that region is contracting away from the centerline and, as such, may be associated with dyskinesia.

In a third embodiment, of the second embodiment, the series of cuts comprises at least a cut along the vertical long axis and the horizontal long axis through the left ventricle.

In a fourth embodiment, of the second or third embodiment, the method further comprises obtaining at least six cuts of the heart.

In a fifth embodiment, of the second, third, or fourth embodiment, the centerline corresponds to the intersection of the series of cuts.

In a sixth embodiment, of any of the first through fifth embodiment, the method further comprises analysing the plurality of images and determining the endocardial border outlining the dimensions of the heart in each of the plurality of images.

In a seventh embodiment, of the sixth embodiment, obtaining an inward displacement measurement of at least one region of the heart comprises determining the position of the endocardial border of the heart in diastole and systole.

In an eight embodiment, of the sixth or seventh embodiment, the method further comprises placing at least one marker on the endocardial border, wherein obtaining an inward displacement measurement of at least a region of the heart based on the difference between the heart wall position in different images of the plurality of images comprises determining movement of the marker between respective images of the plurality of images.

In a ninth embodiment, of the eighth embodiment, assessing the regional contractility of various regions of the heart wall motion based on the inward displacement measurement comprises averaging the determined movement of a plurality of markers.

In a tenth embodiment, of the eighth or ninth embodiment or the second embodiment, or any embodiment dependent thereon, obtaining an inward displacement measurement of at least a region of the heart based on the difference between the heart wall position in different images of the plurality of images comprises determining movement of the marker relative to the centerline between respective images of the plurality of images.

In an eleventh embodiment, of any of embodiments one through ten, wherein at least one of the images of the plurality of images corresponds to the heart at end systole and at least another of the images of the plurality of images to the heart at end diastole, and wherein obtaining an inward displacement measurement of at least a region of the heart based on the difference between the heart wall position in different images of the plurality of images comprises determining the displacement of the region of the heart between end systole and end diastole.

In a twelfth embodiment, of any of embodiments one through eleven, assessing the regional contractility of various regions of the heart wall motion based on the inward displacement measurement comprises comparing the inward displacement measurement to a pool of normal subjects.

In a thirteenth embodiment, of any of embodiments one through twelve, the graphical representation comprises an indication of the percentage of inward displacement compared to a normal heart.

In a fourteenth embodiment, of any of embodiments one through thirteen, the graphical representation is in the form of a bullseye chart.

In a fifteenth embodiment, of any of embodiments one through fourteen, further comprising determining areas of the heart that are suitable for therapeutic treatment by ventricular reconstruction by means of anchor deployment based on the assessed regional contractility; and/or suitable for injection of hydrogels within the myocardium based on the assessed regional contractility.

In a sixteenth embodiment, a computer-implemented method is provided for monitoring heart function for treatment of congestive heart failure and other conditions. The method comprises obtaining a plurality of images of a patient's heart or a portion thereof and obtaining an inward displacement measurement of at least a region or segment of the heart based on the difference in the heart wall position between different images of the plurality of images to provide an indication of the regional contractility of the heart wall motion based on the inward displacement measurement. The method also comprises providing a graphical representation of the regional contractility of the heart wall for determining regions or segments of the heart that are suitable or eligible for therapeutic treatment. Any of embodiments two through fifteen may be practiced in view of the sixteenth embodiment.

In a seventeenth embodiment, a computer-implemented method is provided for monitoring heart function for treatment of congestive heart failure and other conditions. The method comprises obtaining a plurality of images of a patient's heart or a portion thereof and obtaining an inward displacement measurement of at least a region of the heart based on the difference in the heart wall position between different images of the plurality of images. The method also comprises assessing the regional contractility of the heart wall motion based on the inward displacement measurement. The method further comprises determining areas of the heart that are suitable for therapeutic treatment by ventricular reconstruction by means of anchor deployment based on the assessed regional contractility, and/or determining areas of the heart that are suitable for injection of hydrogels within the myocardium based on the assessed regional contractility. Any of embodiments two through fifteen may be implemented as a method in view of the seventeenth embodiment.

In an eighteenth embodiment, of any of embodiments one through seventeen, the method further comprises determining that the heart is not suitable or eligible for therapeutic treatment.

According to certain example embodiments, a method for monitoring heart function to assess for treatment of congestive heart failure and other conditions is provided. The method includes processing a plurality of images of a left ventricle of a heart of a subject, the plurality of images acquired at different points in time in which the heart beats. The method also includes calculating, using at least one hardware processor of a computing system, multiple displacement values that each correspond to an amount by which a corresponding portion of the endocardial border of the left ventricle of the heart displaces towards a centerline of the left ventricle. The method further includes generating and displaying, on a display that is coupled to the computing system, a graphical representation that illustrates the contractility of the left ventricle based on the calculated multiple displacement values.

This Summary is provided to introduce a selection of concepts that are further described below in the Detailed Description. This Summary is intended neither to identify key features or essential features of the claimed subject matter, nor to be used to limit the scope of the claimed subject matter; rather, this Summary is intended to provide an overview of the subject matter described in this document. Accordingly, it will be appreciated that the above-described features are merely examples, and that other features, aspects, and advantages of the subject matter described herein will become apparent from the following Detailed Description, Figures, and Claims.

### BRIEF DESCRIPTION OF THE DRAWINGS

These and other features and advantages will be better and more completely understood by referring to the following detailed description of example non-limiting illustrative embodiments in conjunction with the drawings of which:

FIG. 1A shows three-dimensional image of a heart, with illustrative planar “cuts” according to certain examples;

FIG. 1B shows the 6 different planar cuts that are shown in FIG. 1A;

FIG. 1C is a view of a three-dimensional model of a heart defined with an endocardial border with displacement vectors towards a center line according to certain example embodiments;

FIG. 1D graphically shows the calculation of inward displacement according to certain example embodiments;

FIG. 1E graphically shows the relationship between a heart and segments of a bullseye chart according to certain example embodiments;

FIGS. 2 and 3 are example images of the left ventricle of a human heart according to certain examples;

FIG. 4 shows an example image of the basal inferior of a heart according to certain examples;

FIG. 5 is an example graphical representation of the basal inferior of the heart shown in FIG. 4 according to certain examples;

FIG. 6 shows an exemplary image of an apical chamber view of a heart according to certain examples;

FIG. 7 is an example graphical representation of the heart view shown in FIG. 6 according to certain examples;

FIG. 8 shows an exemplary image of another apical chamber view of a heart according to certain examples;

FIG. 9 is an example graphical representation of the heart view shown in FIG. 8 according to certain examples;

FIG. 10 shows an exemplary 17-segment bullseye chart according to certain examples;

FIG. 11 shows a 17-segment bullseye chart with example inward displacement values of corresponding heart regions according to certain examples;

FIG. 12A shows a 17-segment bullseye chart that has been populated with inward displacement values according to certain examples;

FIG. 12B shows a 17-segment bullseye chart that has been populated with percentage inward displacement values according to certain examples;

FIGS. 13A-13G are cross sectional images of a heart showing a path of markers during contraction and expansion of the heart according to certain examples;

FIGS. 14A-14F are cross sectional images of a heart with a defined heart wall that has vectors that show movement directions of the heart wall during contraction and expansion of the heart according to certain examples;

FIG. 15 is a flow chart of an example process for performing and determining whether therapeutic treatment should be performed according to certain example embodiments; and

FIG. 16 shows an example computing device that may be used in some embodiments to implement features described herein.

### DETAILED DESCRIPTION

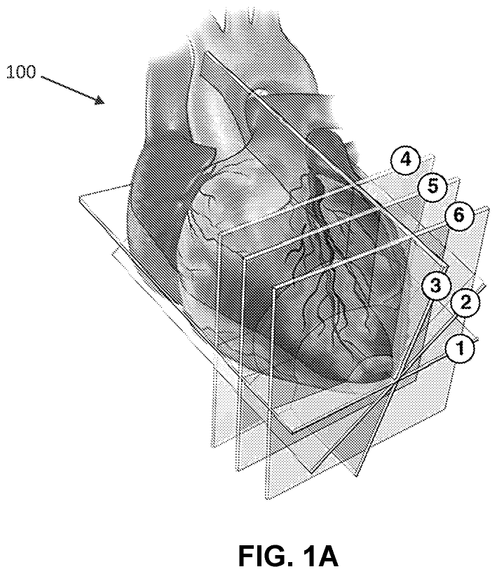

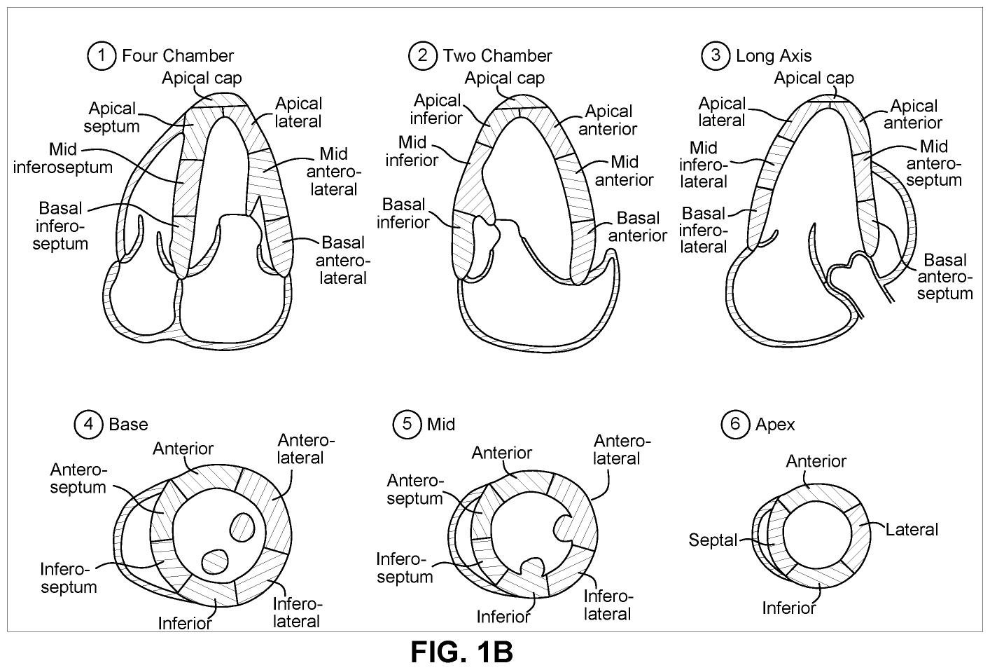

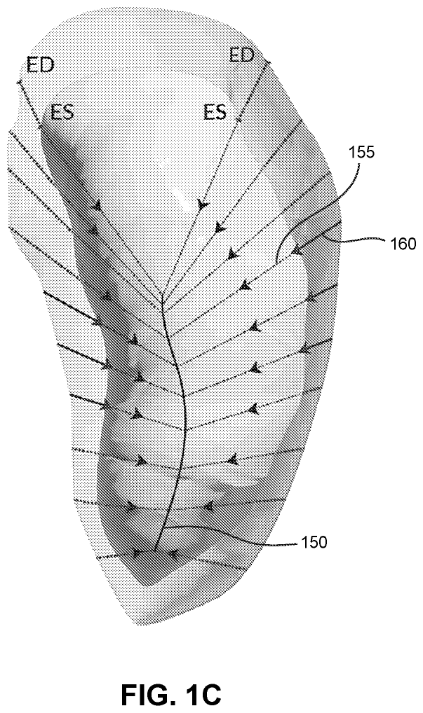

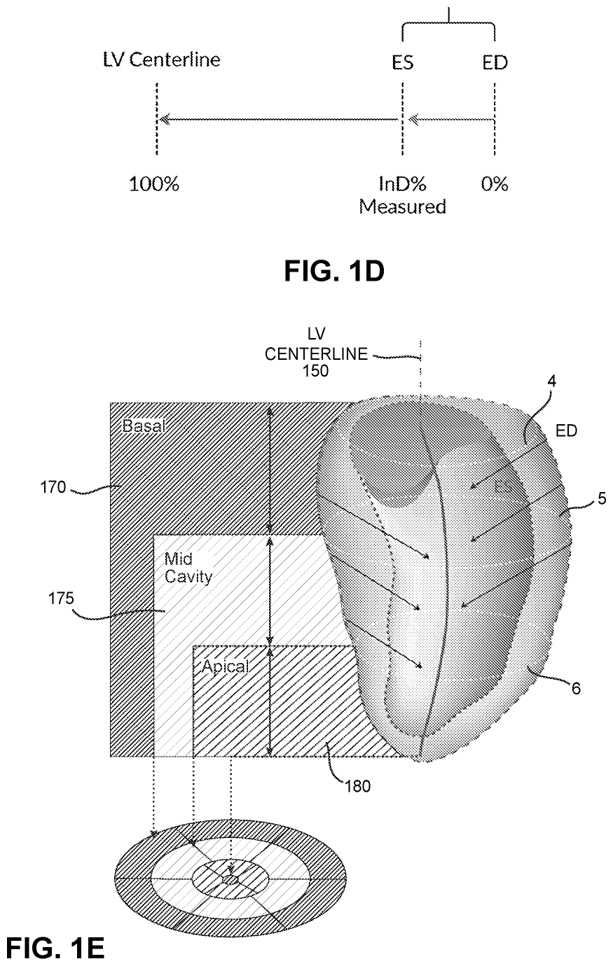

FIG. 1A shows three-dimensional planar “cuts” of the heart 100, labeled as cuts 1-6. These are used to measure the inward displacement as discussed herein. At the intersection of these “cuts” is a centerline, as discussed further below. FIG. 1B details the resulting views which are achieved from each of the cuts 1-6 respectively. For example, at cut 1, a four-chamber view of the heart is shown. Using these cuts, a three-dimensional representation of the movement of the heart at various aspects can be measured. In various implementations of the present disclosure, 3 of these cuts may be used to create a three-dimensional implementation of the heart. For example, 3 long-axis views of the heart—vertical long axis, horizontal long axis and 3 chamber views (e.g., cuts 1-3 of FIG. 1A) may provide sufficient visual representation of the heart.

According to various implementations of the inward displacement method, as described herein, a centerline may be created (for example, in the LV or RV). This centerline may correspond with the one of the cuts (1-6) described in FIG. 1A. As the heart beats, and thus expands and contracts, a distance may be measured from the centerline to the end diastole and from the centerline to end systole. By subtracting these values, a displacement value may be assigned or associated with the assessed displacement. For example, a positive number between the end diastole and end systole may mean that the segment of the heart is contracting inwards. A number which is zero, or approximately zero, may mean, or be associated with, akinesia. A negative number may mean that segment is contracting away from the centerline and, as such, may be associated with dyskinesia.

FIG. 1C shows a three-dimensional model of a heart at the end diastole (ED) and the end systole (ES). Shown in FIG. 1C are vector lines (155) that represent the direction and magnitude of the movement (160) from ED to ES towards a center line 150.

FIG. 1D illustrates the relationship between ED, ES, and a centerline of the LV. More specifically, a value InD (inward displacement) is calculated as the difference between ED and ES. Different InD values can be calculated, defined, or obtained for each of multiple points (referred to as markers herein) along the endocardial border of the LV. The InD values can be represented by the component of the displacement vector towards the centerline of the LV. In some examples, InD is measured in millimeters and expressed as a percentage value, with 0% representing no contraction between ES and ED and 100% corresponding to a theoretical limit in which the LV shrinks to a zero volume (e.g., the endocardial border has contracted to the centerline of the LV). As discussed herein, negative percentage values may correspond to a lack of contract by the LV.

FIG. 1E shows graphical representations of the left ventricle at ES and ED. The arrows shown in FIG. 1E illustrate the direction of contraction from ED to ES. This figure also illustrates the relationship between regions of the left ventricle of the heart (the Basal region 170, mid cavity region 175, and the apical region 180) that correspond to cuts 4, 5, and 6 that are illustrated in FIG. 1A. These regions can be mapped onto the various segments of a 17-segment bullseye chart—as discussed in connection with FIG. 10.

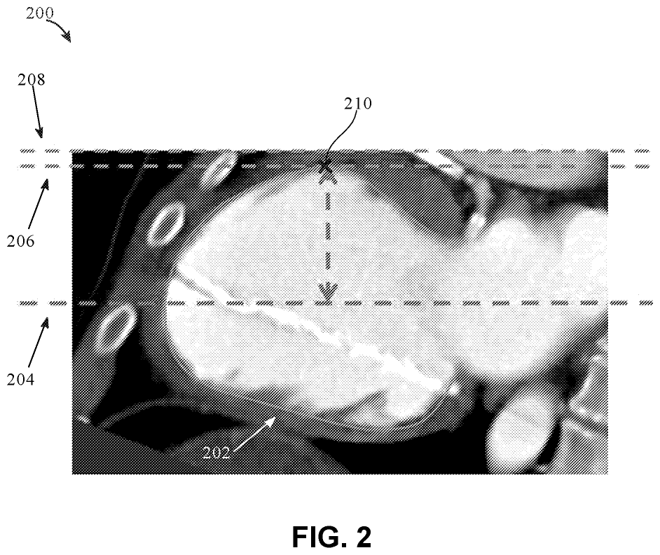

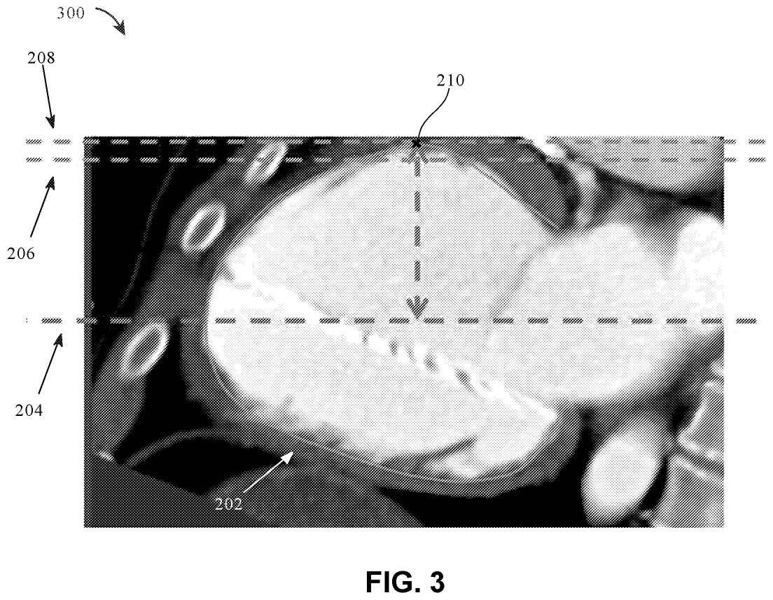

FIGS. 2-3 shows exemplary magnetic resonance imaging (MRI) views (200, 300) of a left ventricle (LV) of a human heart. These views may be stored on computing device 1600 and/or displayed on display device 1612. In view 200, the heart is shown in end systole, and in view 300 the heart is shown in end diastole. The views (200, 300) of the heart are shown with an endocardial border 202 outlining the dimensions of the heart superimposed on the MRI views at the end systole and end diastole. Further the views (200, 300) include a centerline 204 superimposed on the MRI.

As discussed in greater detail below, various techniques may be used in defining or assessing the endocardial border 202. In some examples, one or more markers (e.g., 210) may be used to define the endocardial border 202. For example, a user may define a plurality of markers and these markers may be used to define the endocardial border 202. In some embodiments, the centerline may be automatically defined. For example, the centerline may be automatically defined as a function of where the markers have been placed/defined by the user on the images. In other embodiments, the centerline may be manually defined by a user.

According to various implementations of the inward displacement method, inward displacement can be assessed for the LV shown in FIGS. 2 and 3. At view 200, where the heart is shown at end systole a measurement may be taken between the centerline 204 to an end systole height 206 associated with a marker 210 on the endocardial border 202. At view 300, where the heart is shown at end diastole, an additional measurement may be taken between the centerline 204 and an end diastole height 208 associated with the same marker 210 on the endocardial border 202. The end systole height 206 may then be subtracted from the end diastole height 208 to provide a difference corresponding to the displacement of the heart at the location of marker 210. A similar analysis may be performed (simultaneously or sequentially) for a plurality of marker locations along the endocardial border 202. The method may then be performed using other cuts, as described in connection with, for example, FIG. 1A, to provide a three-dimensional representation of the displacement of various portions of the heart, or portions of a particular heart chamber, as described and discussed further below.

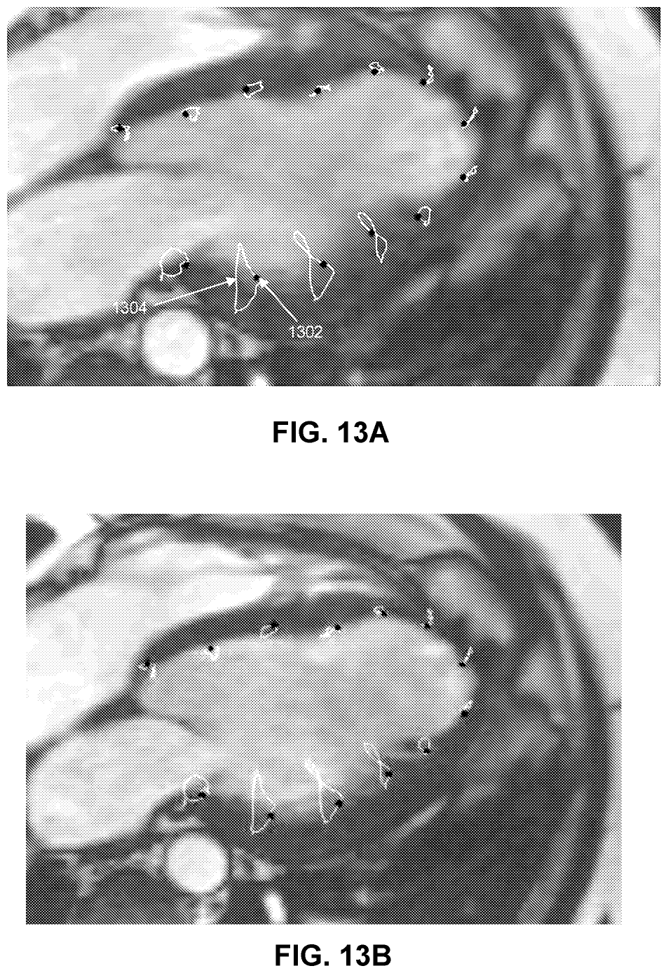

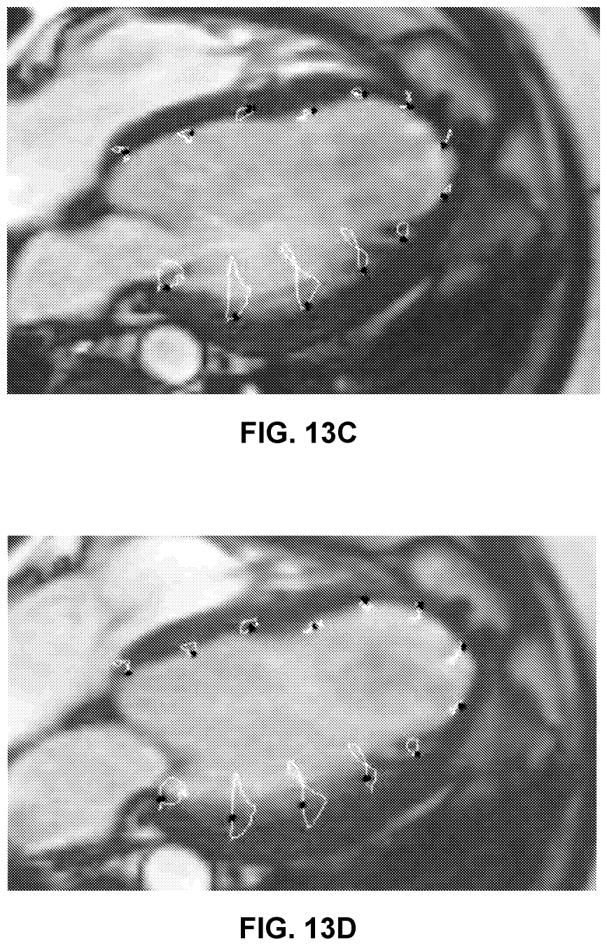

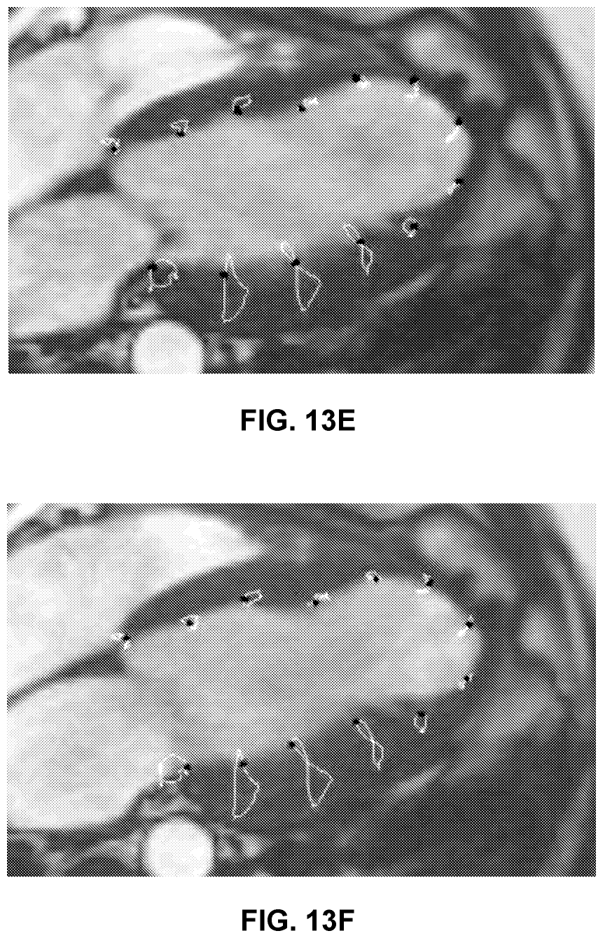

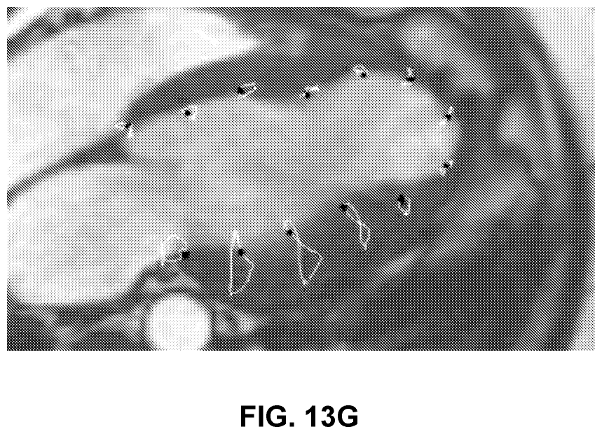

In some examples, the markers may be defined or set based on a user defining a location of each or any of the markers on any or each of the images of the heart (e.g., the LV or RV). For example, a user (e.g., doctor, physician, medical technician, or the like) may use an input device 1614, such as a touch screen or a mouse, to provide input that defines where within any or each or the images of the heart the marker is located (e.g., along the endocardial border of the LV). In some examples, markers may be automatically positioned within an image and then, as needed, manually adjusted by a user. Each marker may then be tracked across the different images of the heart to show how that marker moves between ED and ES. Illustrative examples are shown in FIGS. 13A-13G that show a path each of the markers that have been set take during the contraction/expansion process of the heart.

Inward displacement may provide advantages of accuracy in showing which segments of the heart (or specifically the LV or RV) have normal contractility, hypokinesia, akinesia, dyskinesia, and the like. Inward displacement allows direct comparison between segments and an objective determination of which segments are akinetic or dyskinetic, which may aid in determining areas of the heart that are suitable or eligible for therapeutic treatment, such as ventricular reconstruction by means of BioVentrix Revivent TC anchor deployment, Ancora Heart Accucinch® Ventricular repair system, injection of hydrogels within the myocardium, stem cell therapies within the damaged myocardium, etc. Inward displacement also increases the objectiveness of scar determination, mainly in CT, where late gadolinium enhancement is not available. The identification of scar tissue may aid in determining a proper placement of heart anchors for use in reconstructing the ventricle. Inward displacement accurately represents by a quantifiable number the contractility of a given segment, making possible intra and inter-heart comparisons. In various aspects results using inward displacement may be based at least partially on the comparison with a pool of normal subjects.

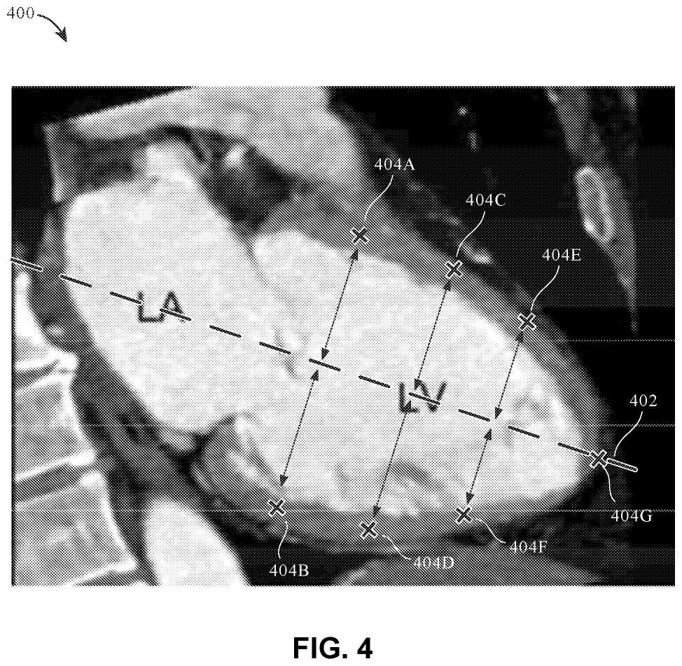

FIG. 4 shows an exemplary MRI image 400 of the basal inferior (segment 4) of the heart, having a left ventricle (LV) and left atrium (LA). Superimposed over the image 400 is a centerline 402, which transects the heart along an axis from the apex. A plurality of markers 404 may be positioned along the outside of the heart wall for measurement of inward displacement. For example, the markers 404 may include a basal anterior maker 404A, basal inferior marker 404B, mid anterior marker 404C, mid inferior marker 404D, apical anterior marker 404E, apical inferior marker 404F, and apex marker 404G. In other embodiments more or fewer markers 404 may be included in the left ventricle. As discussed above, inward displacement values may be measured at each of the markers 404 by measuring the position of the respective markers 404 from the centerline 402 between the end diastole and end systole.

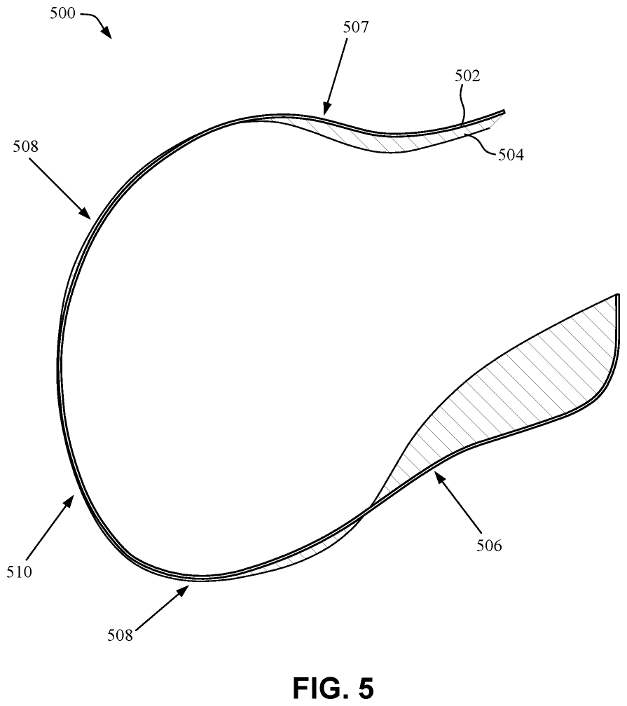

FIG. 5 shows an exemplary graphical representation 500 of the basal inferior (segment 4) of the heart corresponding with the MRI image 400 of FIG. 4. The graphical representation 500 may be produced by aggregating or collecting the data or measurements of the various markers as illustrated in FIG. 4. The data or measurements may be collected over a given amount of time and an average of the data, or end values, may be plotted or graphically displayed. The data from the discrete locations may be used to define a continuous surface or boundary that is associated with the wall of the heart, or the endocardial border 202 may be used to plot the continuous surface or boundary. As illustrated in FIG. 5, the continuous surface or boundary of the heart may be graphically illustrated in addition to, or as an alternative to, the discrete locations associated with the various markers 404. The use of the graphical representation 500 may enable a physician to quickly and accurately assess a functioning of the heart. In the graphical representation 500, the heart position, and more specifically the continuous surface or boundary associated with the heart wall, is shown at end diastole 502 and end systole 504. Inward displacement allows for the position of the heart at end diastole 502 and end systole 504 to be compared, and changes between the heart's shape at end diastole 502 and end systole 504 can be mapped. For example, the heart may have regions with a more normal or typical contractility 506 and/or regions that exhibit hypokinesia 507, akinesia 510, and/or dyskinesia 508.

An amount of displacement or contraction of the various heart segments or regions may also be graphically illustrated in FIG. 5 as the shaded/crosshatched area between the end diastole 502 and end systole 504. The display of the shaded or crosshatched area may be adjusted to graphically display a condition of the heart wall displacement. For example, the shading or crosshatch pattern may be adjusted based on normal contractility, hypokinesia, akinesia, dyskinesia, and the like of the heart wall. This graphical illustration may aid in quickly identifying or determining portions of the heart that appear to have a more normal or typical function in comparison with a healthy heart or other portions of the heart. For example, the inward displacement can be displayed for each segment of the heart. The data in the table below indicates that the apical region of the heart at the septum and anterior is akinetic and the mid ventricle is hypokinetic at both the anterior and inferior septum, which may warrant further investigation or indicate that one or more of these portions of the heart are areas to treat with a therapeutic procedure. Whereas the basal region of the heart has a more normal function. The results may be expressed in millimeters, as percentage of the normal values, or as a score integrating, in example, LV volumes.

The graphical illustration may also enable a physician to determine areas of the heart that are eligible for a therapeutic procedure, or that require further investigation to determine the applicability of a therapeutic procedure.

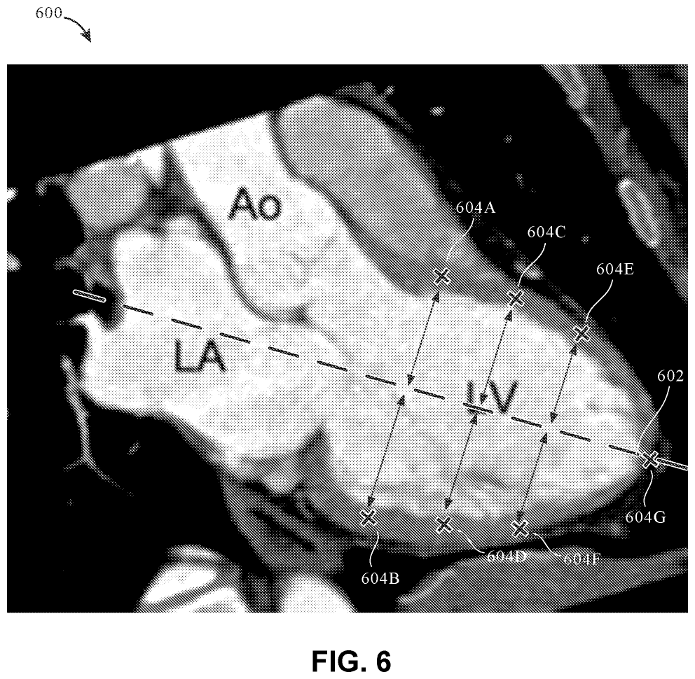

FIG. 6 shows an exemplary MRI image 600 of the apical 3 chamber view of the heart, having a left ventricle (LV), left atrium (LA), and aorta (Ao). Superimposed over the image 600 is a centerline 602, which transects the heart along an axis from the apex. A plurality of markers 604 may be positioned along the outside of the heart wall for measurement of inward displacement. For example, the markers 604 may include a basal anteroseptum maker 604A, basal inferolateral marker 604B, mid anteroseptum marker 604C, mid inferolateral marker 604D, apical anterior marker 604E, apical inferior marker 604F, and apex marker 604G. In other embodiments more or fewer markers 604 may be included in the left ventricle. As discussed above, inward displacement values may be measured at each of the markers 604 by measuring the position of the respective markers 604 from the centerline 602 between the end diastole and end systole.

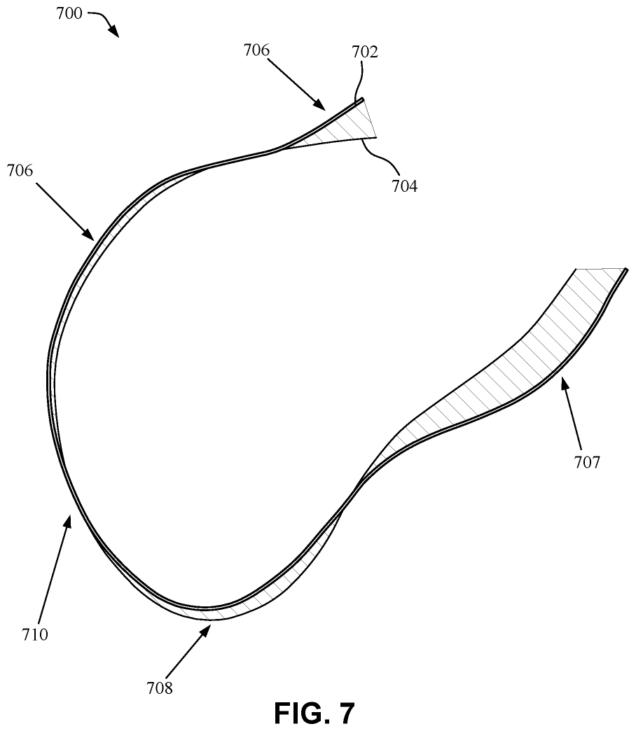

FIG. 7 shows an exemplary graphical representation 700 of the apical 3 chamber view of the heart from FIG. 6. In the graphical representation 700, the heart position is shown at end diastole 702 and end systole 704. Inward displacement allows for the position of the heart at end diastole 702 and end systole 704 to be compared, and changes between the heart's shape at end diastole 702 and end systole 704 to be mapped. For example, the heart may have regions with normal contractility 706, hypokinesia 707, dyskinesia 708, akinesia 710.

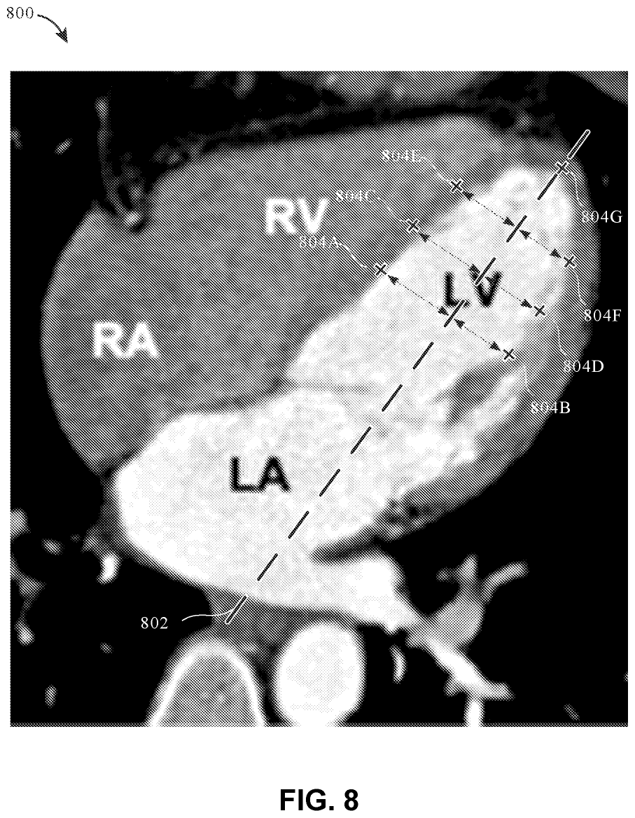

FIG. 8 shows an exemplary MRI image 800 of the apical 4 chamber view of the heart, having a left ventricle (LV), right ventricle (RV), left atrium (LA), and right atrium (RA). Superimposed over the image 800 is a centerline 802, which transects the heart along an axis from the apex. A plurality of markers 804 may be positioned along the outside of the heart wall for measurement of inward displacement. For example, the markers 804 may include a basal inferoseptum maker 804A, basal anterolateral marker 804B, mid inferoseptum marker 804C, mid anterolateral marker 804D, apical septum marker 804E, apical lateral marker 804F, and apex marker 804G. In other embodiments more or fewer markers 804 may be included in the left ventricle. As discussed above, inward displacement values may be measured at each of the markers 804 by measuring the position of the respective markers 804 from the centerline 802 between the end diastole and end systole.

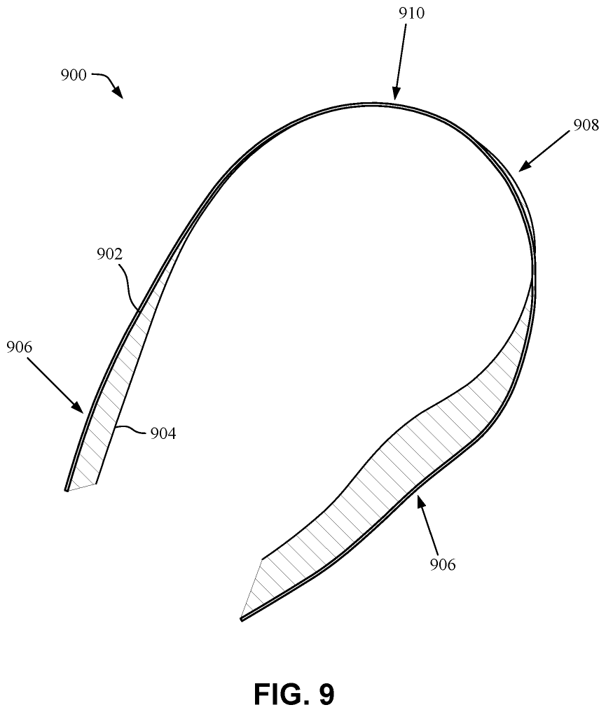

Referring to FIG. 9, an exemplary graphical representation 900 is shown of the apical 4 chamber view of the heart from FIG. 8. In the graphical representation 900, the heart position is shown at end diastole 902 and end systole 904. Inward displacement allows for the position of the heart at end diastole 902 and end systole 904 to be compared, and changes between the heart's shape at end diastole 902 and end systole 904 can be mapped. For example, the heart may have regions with normal contractility 906, diskinesia 908, akinesia 910, and the like. Similar graphical representations may be produced for any desired segment, or chamber, of the heart in order to measure or assess the inward displacement of that segment, or chamber, of the heart and thereby identify or assess the condition of the heart. For example, multiple “cuts” may be taken of a portion of the heart to determine the inward displacement of those cuts and thereby identify contractile tissue and scar tissue that may define a boundary along which one or more heart anchors may be delivered to reconstruct the heart.

The images shown in FIGS. 4, 6, and 8 may be stored in memory device 1604 of computing device 1600 and may be displayed to users on display device 1612 where users can use input device(s) 1614 to annotate such images (e.g., with markers and/or a centerline, etc.). The graphical representations shown in FIGS. 5, 7, and 9 may be stored in memory device 1604 of computing device 1600 and may be displayed to users on display device 1612. Physicians may use the displayed images or representations to assess a person's heart according to the techniques described herein.

As shown in Table 1, below, inward displacement values calculated, measured, or determined from markers 404, 604, and 804 can be computed and compiled to create a data set representing a three-dimensional data-set of the inward displacement of the heart.

TABLE 1
Exemplary Inward Displacement Values
Inward Displacement

| | | |
|---|---|---|
| Apical septum (604E, 804E) | 0 | mm |
| Apical lateral (604F, 804F) | −2 | mm |
| Apical inferior (404F) | −1 | mm |
| Apical anterior (404E) | 0 | mm |
| Mid anterior (404C) | −2 | mm |
| Apex (404G, 604G, 804G) | −2 | mm |
| Mid anteroseptum (604C) | −4 | mm |
| Mid inferoseptum (804C) | −3 | mm |
| Mid inferior (404D) | −2 | mm |
| Mid inferolateral (604D) | −1 | mm |
| Mid anterolateral (804D) | 0 | mm |
| Basal anterior (404A) | +4 | mm |
| Basal anterospetum (604A) | +3 | mm |
| Basal inferoseptum (804A) | +3 | mm |
| Basal inferior (404B) | +4 | mm |
| Basal inferolateral (604B) | +4 | mm |
| Basal anterolateral (804B) | +4 | mm |

The data compiled in connection with/from the markers 404, 604, and 804 can be broken into numerical averages representing larger areas of the LV. For example, raw values of inward displacement can be averaged for each of the basal, mid, and apical regions of the LV. Specifically, the inward displacement values for the basal markers (404A, 604A, 804A, 404B, 604B, and 804B) can be averaged to provide an average inward displacement of the basal region. Similarly, the mid and apical regions could be averages to provide average inward displacements of these respective regions. Table 2 below, in the column titled “Segmental Average (mm)”, provides exemplary results of averaging such inward displacement values for each region. These values may, for example, provide further insight of the overall health and function of the LV at each region (i.e., basal, mid, and apical regions). In various embodiments, other averages may be provided which present other insight to the results of the inward displacement method.

In various embodiments of the present disclosure, certain values obtained from various markers 404, 604, 804 may be excluded from the averages. For example, inward displacement from the basal anteroseptum marker 604A, basal inferoseptum marker 804A, mid anteroseptum marker 604C, and/or mid inferoseptal 804C may be excluded from the average values of the basal region and mid region. In some instances, these exclusions may provide additional insight and, in some cases, more accurate representations of the function of the LV.

In various embodiments of the present disclosure, data compiled from the markers 404, 604, and 804 may be compared to data from other patients, for example from a dataset of healthy or normal hearts (“the dataset”). From this comparison a percentage of normal function can be generated for the subject heart. These comparisons can be percentages of normal function for each of the markers 404, 604, 804. For example, in Table 2, the column titled (% of InD compared to Normal Subjects (%)) shows the percent of inward displacement compared to a database of normal or healthy hearts. Thus, in Table 2, the measured value of inward displacement for the patient at the basal anterior (e.g., from basal anterior marker 404A) was 29.211% of a normal statistical heart. In various embodiments, inward displacement of the average of multiple markers may be compared to the same multiple markers from the dataset. For example, an average of inward displacement from the basal markers (404A, 604A, 804A, 404B, 604B, and 804B) can be compared to an average of the basal values in the dataset. As shown in Table 2 in the column titled “Segmental Average of % of InD (%)”, for example, the average segmental inward displacement for the basal markers (404A, 604A, 804A, 404B, 604B, and 804B) is 28.8% of a normal statistical heart. Similar comparison may be made for the mid and apical regions of the heart as well. The data provided in Table 2 may enable a quick assessment of the function of the various regions of the heart. For example, based on the data in Table 2, a physician may determine that the basal region of the heart is contracting at a more normal level in comparison with the mid and apical regions of the heart, which may aid in determine if one or more regions of the heart are eligible for a therapeutic procedure and/or are areas that need further assessment.

TABLE 2
Averages and Comparisons Of Inward
Displacement with Normal Subjects

| | % InD | | % of InD | Segmental |
|---|---|---|---|---|
| | to LV | Segmental | compared | Average |
| | Centerline | Average | to Normal | of % of |
| Segment | (mm) | (mm) | Subjects (%) | InD (%) |
| 1)basal anterior | 9.167 | 8.2 | 29.211 | 28.8 |
| 2) basal | 8.313 | | 33.373 | |
| anteroseptal | | | | |
| 3) basal septal | 2.745 | | 11.144 | |
| 4) basal inferior | 8.997 | | 29.125 | |
| 5) basal posterior | 12.171 | | 42.571 | |
| 6) basal lateral | 8.675 | | 27.786 | |
| 7) mid anterior | 3.753 | 8.0 | 10.958 | 25.8 |
| 8) mid | 8.351 | | 31.655 | |
| anteroseptal | | | | |
| 9) mid septal | 2.074 | | 7.969 | |
| 10) mid inferior | 11.582 | | 30.536 | |
| 11) mid posterior | 11.185 | | 37.147 | |
| 12) mid lateral | 6.69 | | 21.883 | |
| 13) apical anterior | 1.336 | 3.5 | 4.8 | 16.5 |
| 14) apical septal | 3.872 | | 15.803 | |
| 15) apical inferior | 4.299 | | 20.587 | |
| 16) apical lateral | 3.781 | | 16.325 | |
| 17) apex | 2.081 | | 13.16 | |

FIG. 10 shows an exemplary 17-segment bullseye chart 1000. The bullseye chart 1000 is a graphical method in which the data gathered from the markers 404, 604, 804 can be displayed to better understand the data. The 17-segment bullseye chart is an exemplary industry standard graphical representation, recognized by the American Heart Association, displaying data relevant to the heart. According to various implementations, the outer ring areas of the bullseye chart 1000 can correspond to basal regions of the heart, the secondary ring can correspond to the mid regions of the heart, the inner ring can correspond to the apical regions of the heart, and the center can correspond to the apex of the heart. More specifically, the regions can correspond with the areas of the heart as shown in Table 3, below.

According to various embodiments, data gathered from various markers may be averaged with respect to one another before being populated in a bullseye chart or other graphical representation of the data. For example, as the dimensions of the heart narrow near the apical region and apex of the heart, the dimensions may become restrained such that an average of data gathered from the apical region may be combined and averaged, such as to simplify the data. More specifically, according to various embodiments, the data value generated for the apical septal region 1014 may be an average of the apical anterior marker 604E and the apical septum marker 804E. Similarly, the apical lateral region 1016 may be an average of the apical inferior marker 604F and the apical lateral marker 804F. Further the apex region 1017 may be populated based on an average of apex marker 404G, apex marker 604G, and apex marker 804G to give a more accurate data point representing the true apex of the heart.

TABLE 3
Corresponding Heart Regions on Bullseye Chart

| Bullseye Chart | Corresponding | Exemplary Corresponding |
|---|---|---|
| Region | Heart Region | Markers 404, 604, 804 |
| 1001 | Basal anterior | 404A |
| 1002 | Basal anteroseptal | 604A |
| 1003 | Basal inferoseptal | 804A |
| | (basal septal) | |
| 1004 | Basal inferior | 404B |
| 1005 | Basal inferolateral | 604B |
| | (basal posterior) | |
| 1006 | Basal anterolateral | 804B |
| | (basal lateral) | |
| 1007 | Mid anterior | 404C |
| 1008 | Mid anteroseptal | 604C |
| 1009 | Mid inferoseptal | 804C |
| | (mid septal) | |
| 1010 | Mid inferior | 404D |
| 1011 | Mid inferolateral | 604D |
| | (mid posterior) | |
| 1012 | Mid anterolateral | 804D |
| | (mid lateral) | |
| 1013 | Apical anterior | 404E, |
| 1014 | Apical septal | 604E and 804E |
| 1015 | Apical inferior | 404F, |
| 1016 | Apical lateral | 604F and 804F |
| 1017 | Apex | 404G, 604G, and 804G |

FIG. 11 shows a 17-segment bullseye chart 1100, similar to that of the bullseye chart 1000 of FIG. 10, but with exemplary inward displacement values of the corresponding heart regions (e.g., as shown from Table 3) populated within the chart. As discussed previously, a positive inward displacement value may mean that the segment of the heart is contracting inwards. An inward displacement value which is zero, or approximately zero, may mean, or be associated with, akinesia. A negative inward displacement value may mean that segment is contracting away from the centerline and, as such, may be associated with dyskinesia.

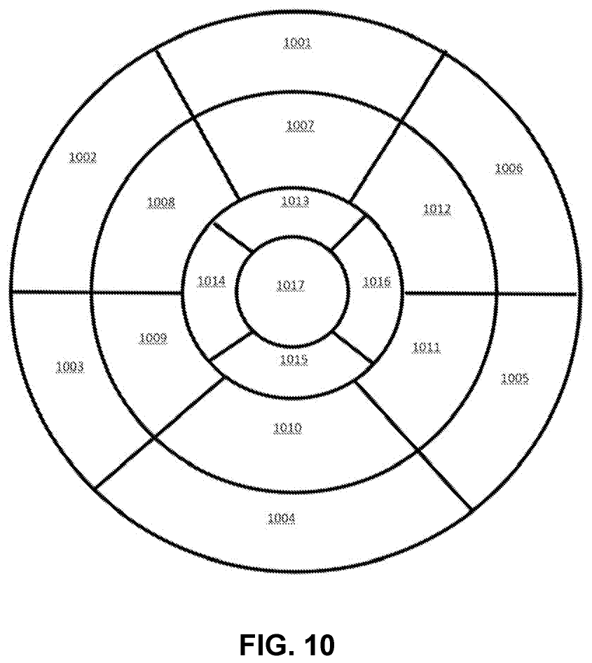

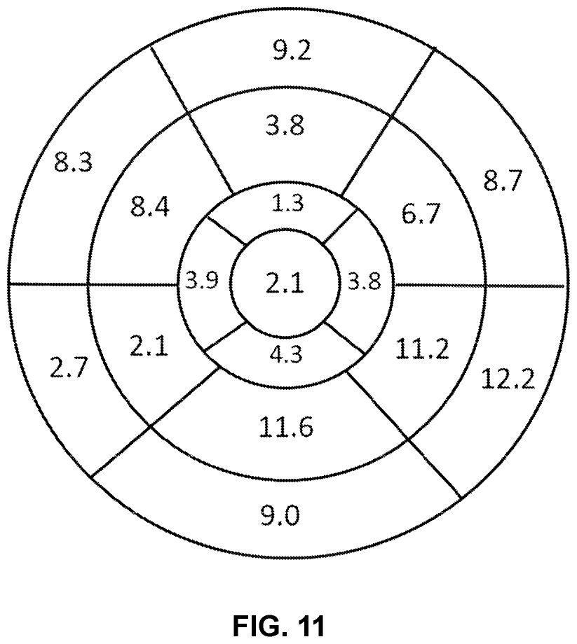

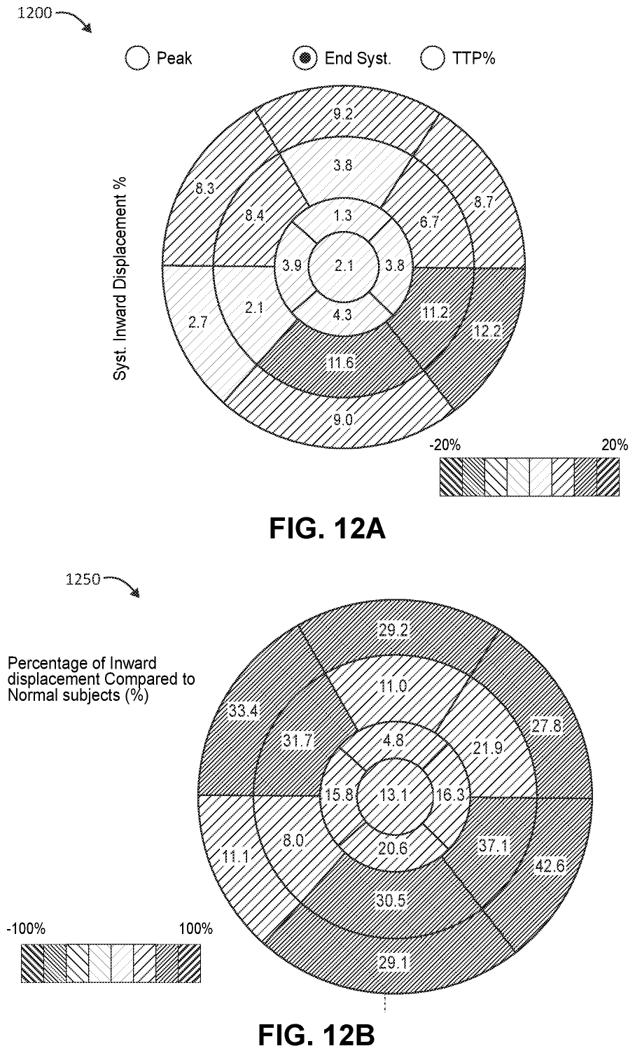

FIG. 12A shows a 17-segment bullseye chart 1200 populated with the values from the inward displacement measurements from Table 2, above. Specifically, the chart 1200 shows the raw values (e.g., not percentage comparisons to inward displacement from other heart images) for each of the 17 segments of the left ventricle. In various embodiments, the bullseye chart 1200 may use graphical representations to show a healthy or unhealthy inward displacement value. For example, the bullseye chart 1200 may be color coded. The colors may be in a scale, such as dark blue, light blue, light red, dark red, where one end of the spectrum represents positive numbers (e.g., red) and the other end of the spectrum represents negative numbers (e.g., blue). In this configuration, a higher (absolute) number will have a darker shading than a lesser (absolute) number. The color-coded segments visually represent how the segments of the various regions are functioning as well as visually representing how the various regions are functioning. For example, darker red colors can be used represent more normal contraction while darker blue colors can be used to represent greater degrees of expansion. Based on this color coding, it can be quickly determined that the inferior and inferolateral (basal posterior) of the basal region and the inferior and inferolateral (mid posterior) of the mid region have a more typically contraction in comparison with the other segments of the heart. In addition, it can be quickly determined that the basal region as a whole (i.e., the outer ring) has a more typical contraction in comparison with the mid and apical regions of the heart.

In FIG. 12B, shows a 17-segment bullseye chart 1250 populated with the values from the inward displacement measurements from Table 2, above. Specifically, the chart 1200 shows the percentage of inward displacement compared to a normal heart. Like chart 1200, chart 1250 may similarly utilize color-coding or other graphical means to distinguish the values of the chart 1250.

In some examples, chart 1200 and/or 1250 may be stored in memory device 1604 of computing device 1600 and may be displayed to users on display device 1612. Such displayed representations of the charts may be used by physicians to assess the health of the heart or portions thereof (e.g., the left ventricle).

While shown herein as a bullseye charts 1200, 1250, other visual displays of data can similarly be used to achieve the same outcome. For example, various graphical displays may provide differing ways to visualize the same data (of the heart in one orientation) taken by the inward displacement method shown herein. Similarly, in some embodiments it may be preferable to have fewer or additional data points shown in a bullseye plot (or other plot) depending on the number of markers and amount of data collected. In some aspects, data from the apex 1017 may not be useful in analyzing heart function, and thus may be excluded, resulting in a 16-segment bullseye chart.

In various embodiments, the bullseye charts 1200, 1250 may be used to compare findings to a similar appearing bullseye chart which contains global strain results from the same patient. This may provide confirmation or redundant findings to ensure or confirm accuracy of the inward displacement results. For example, similar bullseye charts may be prepared which include inward displacement and global strain, respectively. Each of the bullseye charts may be color-coded based on the determination of the results, such that a physician may be able to quickly compare the color-coding of each chart for similarities and differences. In other embodiments, raw data or another visual representation of inward displacement and global strain can be compared.

FIGS. 13A-G illustrate a cross sectional image of a heart, as described in the application, with a plurality of markers 1302 positioned along a wall of the heart chamber (i.e., the left ventricle (LV)). FIGS. 13A-G show the motion of the heart during contraction and relaxation. As the heart contracts and relaxes, a path 1304 of each marker 1302 may be plotted as illustrated. This represents the radial motion of the heart. FIGS. 13A-G show the marker 1302 moving along the plotted path 1304. The motion of the marker 1302, and/or the path 1304, may be used in determining the inward displacement of the heart wall as described in the application.

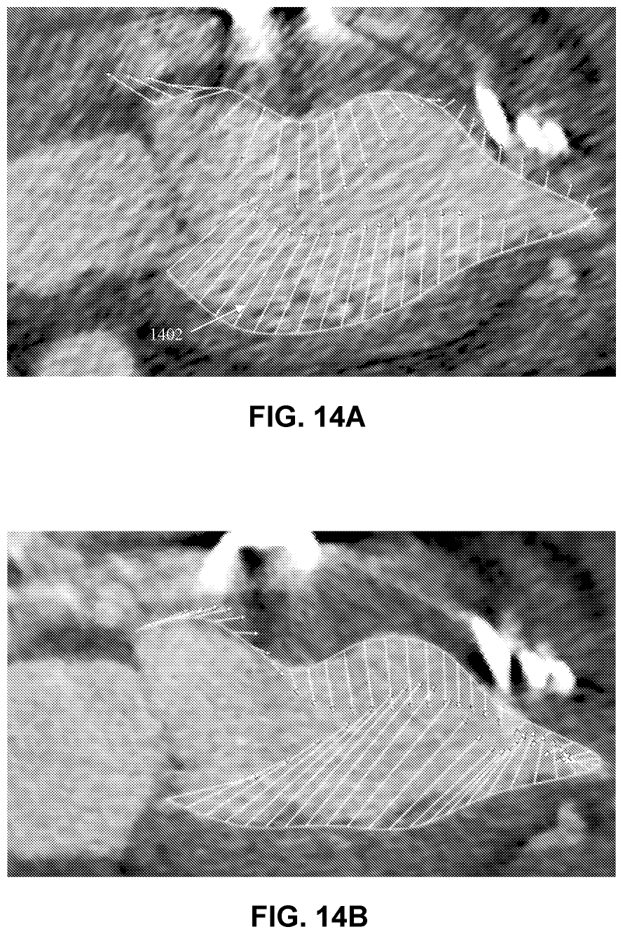

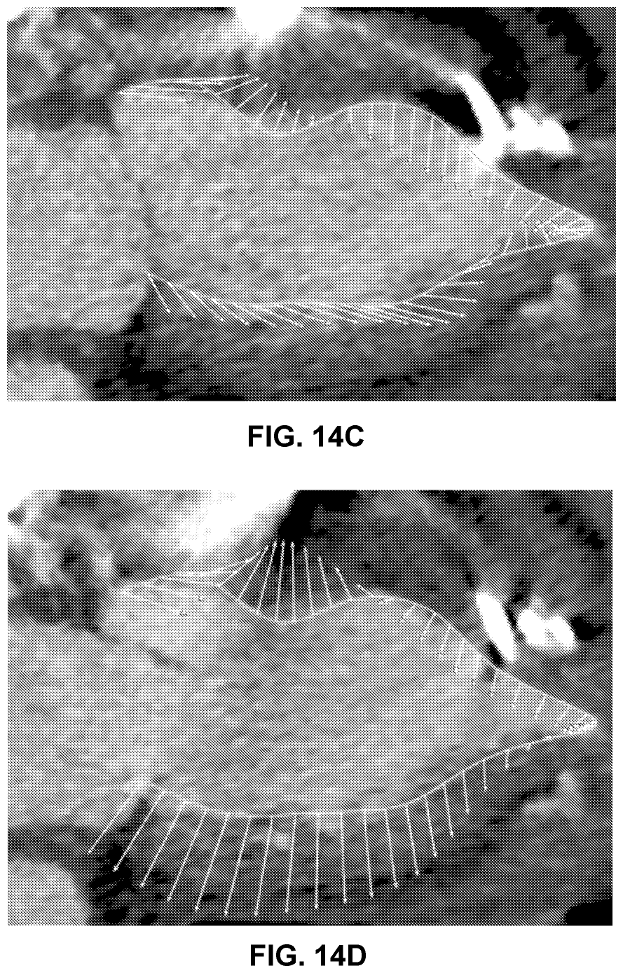

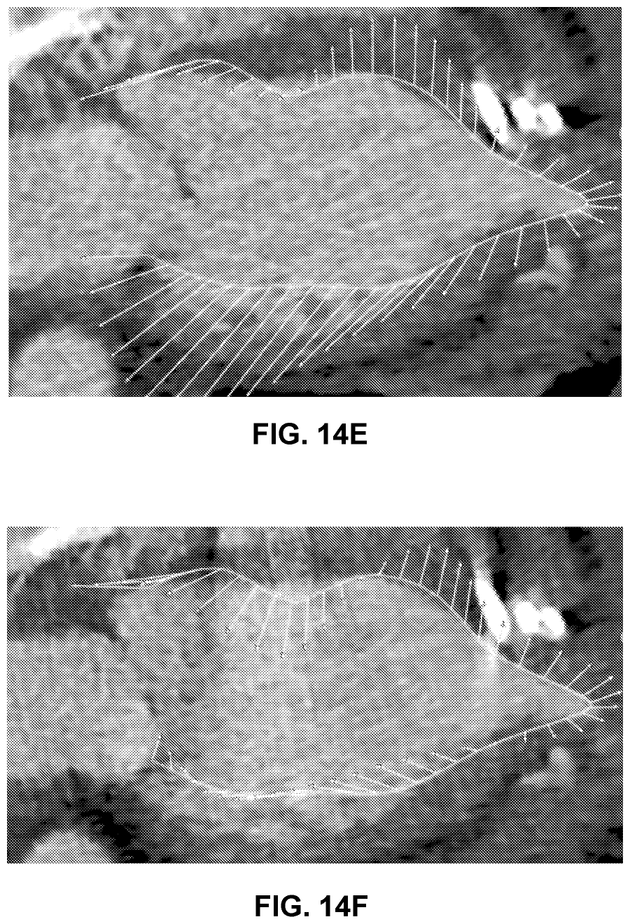

FIGS. 14A-F illustrate a cross sectional image of a heart, as described in the application. Vectors 1402 are superimposed along a wall of the heart chamber (i.e., left ventricle (LV)). The vectors 1402 show the direction that respective portions of the heart wall are moving at any given instance and also indicate the contraction force associated with the respective portions of the heart wall. FIGS. 14A-F show the vectors 1402 during contraction and relaxation of the heart chamber. The vectors 1402, or information associated or gathered therefrom, may be used in determining the inward displacement of the heart wall as described in the application.

The images shown in FIGS. 13A-14F may be stored in memory device 1604 of computing device 1600 and may be displayed to users on display device 1612. The images may be obtained via MRI or CT and then additional processing performed to track the movement of markers across multiple different images (as shown in FIGS. 13A-13G). The vectors shown in FIGS. 14A-14F may be calculated based on the techniques discussed herein and then overlaid, for example, on the images that have been obtained (e.g., MRI or CT images).

The inward displacement method described herein may be useful in providing insight to the health of the heart, and specific function by region of the left ventricle, for example. One use of the inward displacement method disclosed herein is for determining an indication or prediction of success (or likelihood of success) in performing a medical procedure. One such procedure, may involve reconstruction of the heart, or specifically the left ventricle, such as with use of anchors. In these procedures, an implant (anchor) acts to bring opposed walls of the ventricle into contact with one another, such that a portion of the ventricle is excluded or closed off. By reducing the overall size of the ventricle, particularly by reducing the portion of the functioning ventricle chamber defined by scar tissue (such as from congestive heart failure), the heart function may be significantly increased and the effects of disease progression at least temporarily reversed, halted, and/or slowed. Exemplary systems and methods for reconstructing the heart are described in U.S. patent application Ser. No. 17/350,668, filed Jun. 17, 2021, entitled “Trans-Catheter Ventricular Reconstruction Structures, Methods, and Systems for Treatment of Congestive Heart Failure and other Conditions” and U.S. patent application Ser. No. 15/418,152, filed Jan. 27, 2017, entitled “Percutaneous Arterial Access to Position Trans-Myocardial Implant Devices and Methods”, the entire disclosures of which are incorporated by reference herein.

Before performing the anchor implantation, however, it may be desirable to understand the health of the remainder of the ventricle (the residual functionality). For example, if a portion of the ventricle is closed off, the remainder of the ventricle should be healthy enough to provide the necessary blood pumping function. The inward displacement method described herein may, at least in part, provide insight for a physician to understand the capabilities of specific segments of the ventricle, which may enable the physician to determine if the heart is healthy enough to recover from a therapeutic procedures, such as those identified herein. For example, inward displacement may provide data which may suggest that it is not safe for a heart operation to be performed on a given heart. The analysis of the inward displacement may show that the ventricle is not healthy enough to undergo a heart procedure, such as heart anchor implantation. As such, the inward displacement procedure may be used as a screening mechanism to determine suitable candidates for a given heart procedure and/or may be used to assess what procedure among multiple candidate procedures would most effectively treat the heart.

In some examples, the inward displacement procedure may be used to assess and/or provide feedback to a physician as to the results of a heart procedure. For example, prior to a heart procedure, inward displacement may be determined as discussed herein. The heart procedure may then be performed, and a subsequent inward displacement procedure may be performed that allows assessment of the contractility of the heart that has undergone the heart procedure. Such information may be used to validate the efficacy of the heart procedure according to certain example embodiments.

In addition to overall mapping and understanding of ventricle health at various regions, the inward displacement method described herein may also enable a physician to determine where to place anchors for a given procedure. For example, an anchor implantation may target the part of the ventricle that is akinetic. The anchors can be used to close off the akinetic region. Measurements of inward displacement may be used to locate those akinetic portions of the heart, such that a physician may plan a procedure to access those areas and properly position the anchor to maximize effects. Thus, the inward displacement method be used to determine specific parameters for a given heart procedure.

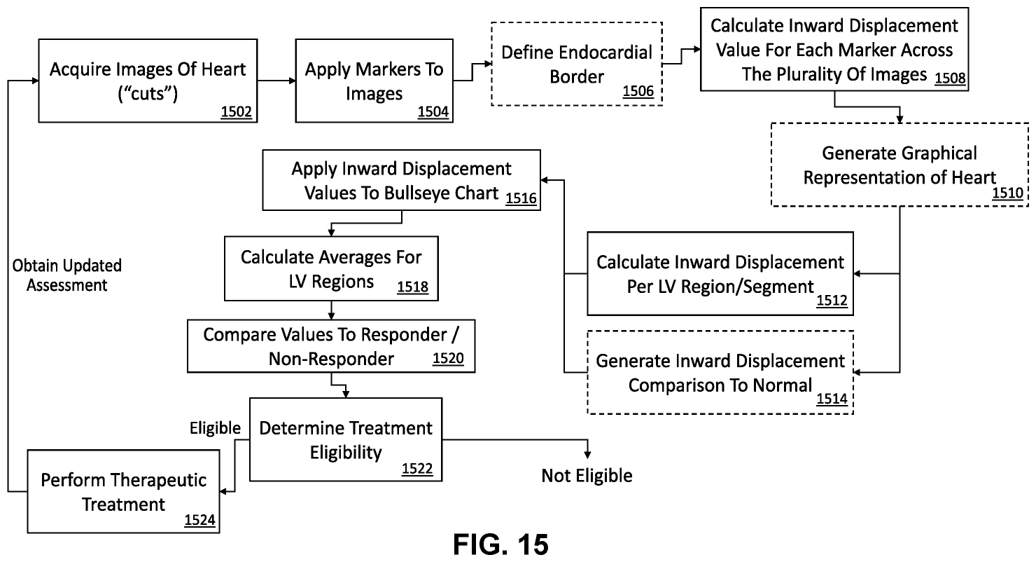

FIG. 15 is a flow chart of an example process for performing and determining whether therapeutic treatment should be performed according to certain example embodiments.

At 1502, a plurality of images of the heart of a patient are acquired. As explained herein, there images may be MRI or CT images and can be acquired as different “cuts” of the heart. For example, two-chamber, three-chamber, and four-chamber views of the heart may be acquired along with 1, 2, or 3 (or more) views of the heart (e.g., views 4, 5, and 6 of FIG. 1A). In some cases, images of the heart may be acquired at different points in time. For example, 10 images may be acquired at different points in time during each heartbeat (e.g., each cardiac cycle). Thus, for example, images may be acquired at 100 ms intervals and be used to present the state of the heart, or portion thereof (such as the LV) over the course of those images. In some examples the images may be acquired by being, emailed or otherwise electronically transferred to a computing system that will be used to perform the processing discussed in FIG. 15. Accordingly, acquiring the images may include receiving them from another computing system or device that generated the images (e.g., an MRI machine or the like).

At 1504, markers may be defined with respect to the images. As discussed herein, these markers may be used to track the movement of the heart during a heartbeat. In some examples, a centerline may also be defined. In some examples, each segment of each region of the heart may have a corresponding marker assigned thereto. Such an approach may allow for a physician getting a three-dimensional view of how the heart (e.g., the LV) expands/contracts over the course of a heartbeat.

At 1506, the endocardial border of the heart wall may be defined or shown based on how the markers have been defined. Such illustrative borders are shown in FIGS. 5, 7, and 9, along with FIG. 2-3.

At 1508, inward displacement values may be calculated for each one of the markers that has been defined in the images. This is shown in, for example, FIGS. 13A-14F, where the vectors in FIG. 14A-F represent the displacement value.

At 1510, a graphical representation of the heart that illustrates the calculated displacement values may be generated and shown to a user. Examples of such graphical representations included FIGS. 5, 7, and 9. It will be appreciated that the bullseye chart is also an example of a graphical representation.

At 1512, a per region and/or per segment calculation of inward displacement values may be calculated. As discussed herein, this may include average two or more values in those regions or segments.

At 1514, in some embodiments, the calculated inward displacement values per marker, per segment, and/or per region may be compared to values for one or more normal functioning hearts.

At 1516, the calculations from 1512 and/or 1514 may be applied to one or more bullseye charts. Illustrative examples of such bullseye charts with values are shown in FIGS. 12A and 12B.

At 1518, the calculations from the various segments and/or each calculated inward displacement value of the LV may be combined to obtain an average for each region of the LV. The regions may include a basal (or base) region, a mid region, and an apical (or apex) region. In some examples, the average for the apical region may not be computed. This averaging of the inward displacement values for these regions will then result in one displacement value for each of the different regions. Thus, for example, one inward displacement value for the basal region and one inward displacement value for the mid region may be computed. The resulting value for these regions may be presented (e.g., displayed on a display screen) to a user to aid in determining whether treatment will be effective for a given patient.

At 1520, the calculated displacement values and/or the averages per region may be compared to data of treatment responder and non-responders. This comparison may further assist in determining whether treatment will be effective for a given patient.

At 1522, a determination may be made as to whether to proceed with therapeutic treatment. In certain examples, this determination may be assisted via the color coding and numbers provided on the bullseye chart. In any event if it is determined that the patient is not eligible, then one or more heart treatment may not be performed (e.g., use of anchors and/or gel-based treatments).

If it is determined that the patient is eligible then, at 1524, one or more therapeutic treatments may be performed as discussed herein.

After treatment, a further assessment of the patient's heart may be performed using the inward displacement techniques discussed herein.

It will be appreciated that any or all of the steps shown in FIG. 15 may be executed on a computer. For example, all of steps 1502-1520 may be executed in whole, or in part, on a computer. Some steps may be entirely executed within a computer (e.g., as software) while others may be supported by execution of software or the like by a computer system (e.g., 1522). In some examples, some of the steps (such as 1522 or 1524) may be performed by a physician or other medical professional (e.g., a physician performing therapeutic treatment). It will be appreciated that even those elements may be supported by the computer operation discussed herein.

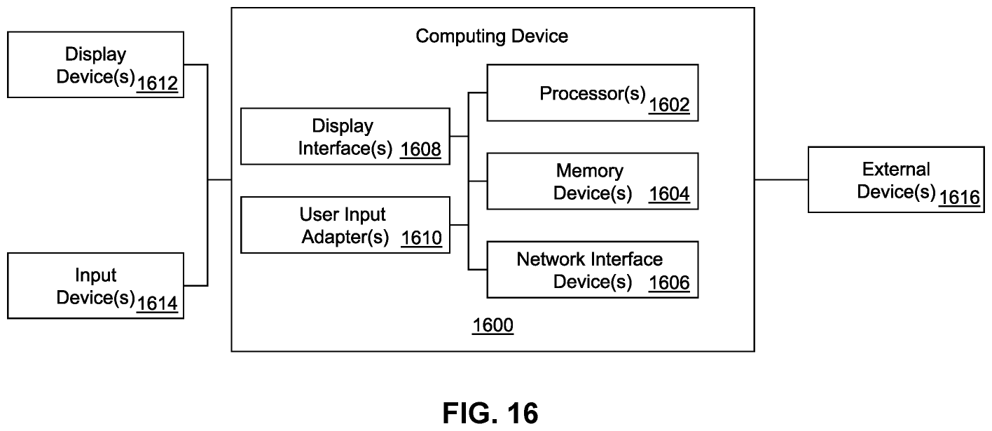

As discussed herein, certain example aspects of the embodiments herein may be performed on or with a computing device. FIG. 16 is a block diagram of an example computing device 1600 (which may also be referred to, for example, as a “computer device,” “computer system,” or “computing system”) according to some embodiments. In some embodiments, the computing device 1600 includes one or more of the following: one or more processors 1602; one or more memory devices 1604; one or more network interface devices 1606; one or more display interfaces 1608; and one or more user input adapters 1610. Additionally, in some embodiments, the computing device 1600 is connected to or include display device(s) 1612. Additionally, in some embodiments, the computing device 1600 is connected to or includes one or more input devices 1614. In some embodiments, computing device 1600 may be connected to one or more external devices 1616. As will be explained below, these elements (e.g., the processors 1602, memory devices 1604, network interface devices 1606, display interfaces 1608, user input adapters 1610, display device 1612, input devices 1614, external devices 1616) are hardware devices (for example, electronic circuits or combinations of circuits) that are configured to perform various different functions for and/or in conjunction with the computing device 1600.

In some embodiments, each or any of the processors 1602 is or includes, for example, a single- or multi-core processor, a microprocessor (e.g., which may be referred to as a central processing unit or CPU), a digital signal processor (DSP), a microprocessor in association with a DSP core, an Application Specific Integrated Circuit (ASIC), a Field Programmable Gate Array (FPGA) circuit, or a system-on-a-chip (SOC) (e.g., an integrated circuit that includes, for example, a CPU, a GPU, and other hardware components such as memory and/or a memory controller (e.g., Northbridge), I/O controller (e.g., Southbridge), networking interfaces, and the like). In some embodiments, each or any of the processors 1602 uses an instruction set architecture such as x86 or Advanced RISC Machine (ARM). In some embodiments, each or any of the processors 1602 is or includes, for example, a graphical processing unit (GPU), which may be an electronic circuit designed to generate images and the like.

In some embodiments, each or any of the memory devices 1604 is or includes a random access memory (RAM) (such as a Dynamic RAM (DRAM) or Static RAM (SRAM)), a flash memory (based on, e.g., NAND or NOR technology), a hard disk, a magneto-optical medium, an optical medium, cache memory, a register (e.g., that holds instructions that may be executed by one or more of the processors 1602), or other type of device that performs the volatile or non-volatile storage of data and/or instructions (e.g., software that is executed on or by processors 1602). In some embodiments, each or any of the memory devices 1604 is removable from the computing device 1600 (such as a USB flash drive, a floppy disk, a Compact disc (CD), and the like). Memory devices 1604 are an example of non-transitory computer-readable storage.

In some embodiments, each or any of the network interface devices 1606 includes one or more circuits (such as a baseband processor and/or a wired or wireless transceiver), and implements layer one, layer two, and/or higher layers for one or more wired communications technologies (such as Ethernet (IEEE 802.3)) and/or wireless communications technologies (such as Bluetooth, WiFi (IEEE 802.11), GSM, CDMA2000, UMTS, LTE, LTE-Advanced (LTE-A), and/or other short-range, mid-range, and/or long-range wireless communications technologies). Transceivers may comprise circuitry for a transmitter and a receiver. The transmitter and receiver may share a common housing and may share some or all of the circuitry in the housing to perform transmission and reception. In some embodiments, the transmitter and receiver of a transceiver may not share any common circuitry and/or may be in the same or separate housings.

In some embodiments, each or any of the display interfaces 1608 is or includes one or more circuits that receive data from the processors 1602, generate (e.g., via a discrete GPU, an integrated GPU, a CPU executing graphical processing, or the like) corresponding image data based on the received data, and/or output (e.g., a High-Definition Multimedia Interface (HDMI), a DisplayPort Interface, a Video Graphics Array (VGA) interface, a Digital Video Interface (DVI), or the like), the generated image data to the display device 1612, which displays the image data. Alternatively or additionally, in some embodiments, each or any of the display interfaces 1608 is or includes, for example, a video card, video adapter, or graphics processing unit (GPU). In other words, the each or any of the display interfaces 1608 may include a processor therein that is used to generate image data. The generation or such images may occur in conjunction with processing performed by one or more of the processors 1602.

In some embodiments, each or any of the user input adapters 1610 is or includes one or more circuits that receive and process user input data from one or more user input devices (1614) that are included in, attached to, or otherwise in communication with the computing device 1600, and that output data based on the received input data to the processors 1602. Alternatively, or additionally, in some embodiments each or any of the user input adapters 1610 is or includes, for example, a PS/2 interface, a USB interface, a touchscreen controller, or the like; and/or the user input adapters 1610 facilitates input from user input devices 1614.

In some embodiments, the display device 1612 may be a Liquid Crystal Display (LCD) display, Light Emitting Diode (LED) display, or other type of display device. In embodiments where the display device 1612 is a component of the computing device 1600 (e.g., the computing device and the display device are included in a unified housing), the display device 1612 may be a touchscreen display or non-touchscreen display. In embodiments where the display device 1612 is connected to the computing device 1600 (e.g., is external to the computing device 1600 and communicates with the computing device 1600 via a wire and/or via wireless communication technology), the display device 1612 is, for example, an external monitor, projector, television, display screen, etc.

In some embodiments, each or any of the input devices 1614 is or includes machinery and/or electronics that generates a signal that is provided to the user input adapter(s) 1610 in response to physical phenomenon. Examples of inputs devices 1614 include, for example, a keyboard, a mouse, a trackpad, a touchscreen, a button, a joystick, a sensor (e.g., an acceleration sensor, a gyro sensor, a temperature sensor, a pressure sensor (e.g., that measures pressure of a gas), a flow sensor (e.g., the measures a rate of gas or liquid flow), and the like), a microphone. In some examples, one or more input devices 1614 generate signals that are provided in response to a user providing an input—for example, by pressing a button, speaking a voice command, or the like. In other examples, one or more input devices generate signals based on sensed physical quantities (e.g., such as force, pressure, temperature, etc.). In some embodiments, each or any of the input devices 1614 is a component of the computing device (for example, a button is provided on a housing that includes the processors 1602, memory devices 1604, network interface devices 1606, display interfaces 1608, user input adapters 1610, and the like).

In some embodiments, each or any of the external device(s) 1616 may include other computing devices (e.g., other instances of computing device 1600) that communicate with computing device 1600. Examples may include a server computer, a client computer system, a mobile computing device, a cloud-based computer system, a computing node, an Internet of Things (IoT) device, a flow generator, etc. that all may communicate with computing device 1600. In general, external devices(s) 1616 may include devices that communicate (e.g., electronically) with computing device 1600. As an example, computing device 1600 may be mobile device communicates with a flow generator or a patient interface device or mask (e.g., examples of external device 1616). Conversely, computing device 1600 may be a flow generator that communicates with server or cloud-based computer system, which are examples of external devices 1616, that provides data and/or software updates to the flow generator.

In various embodiments, the computing device 1600 includes one, or two, or three, four, or more of each or any of the above-mentioned elements (e.g., the processor(s) 1602, memory device(s) 1604, network interface device(s) 1606, display interface(s) 1608, user input adapter(s) 1610, display device(s) 1612, input device(s) 1614). Alternatively, or additionally, in some embodiments, the computing device 1600 includes one or more of: a processing system that includes the processors 1602; a memory or storage system that includes the memory devices 1604; and a network interface system that includes the network interface devices 1606.

The computing device 1600 may be arranged, in various embodiments, in many different ways. As just one example, the computing device 1600 may be arranged such that the processors 1602 include: a multi (or single)-core processor; a first network interface device (which implements, for example, WiFi, Bluetooth, NFC, etc.); a second network interface device that implements one or more cellular communication technologies (e.g., 3G, 4G LTE, CDMA, etc.); memory or storage devices (e.g., RAM, flash memory, or a hard disk). The processor, the first network interface device, the second network interface device, and the memory devices may be integrated as part of the same SOC (e.g., one integrated circuit chip). As another example, the computing device 1600 may be arranged such that: the processors 1602 include two, three, four, five, or more multi-core processors; the network interface devices 1606 include a first network interface device that implements Ethernet and a second network interface device that implements WiFi and/or Bluetooth; and the memory devices 1604 include a RAM and a flash memory or hard disk. As another example, the computing device 5100 may include a SoC with one or processors 5102, plural network interface devices 5106 (e.g., one that uses communicates via a Cellular connection and one that communicates via a Bluetooth connection), memory devices 5104 that include system memory and memory for application programs and other software, a display interface 5108 that is configured to output a video signal, a display device 5112 that is integrated to a housing and layered with a touch screen input device 5114, and multiple input device 5114 such as one or more buttons and/or and one or more sensors.

### Selected Terminology

Whenever it is described in this document that a given item is present in “some embodiments,” “various embodiments,” “certain embodiments,” “certain example embodiments, “some example embodiments,” “an exemplary embodiment,” or whenever any other similar language is used, it should be understood that the given item is present in at least one embodiment, though is not necessarily present in all embodiments. Consistent with the foregoing, whenever it is described in this document that an action “may,” “can,” or “could” be performed, that a feature, element, or component “may,” “can,” or “could” be included in or is applicable to a given context, that a given item “may,” “can,” or “could” possess a given attribute, or whenever any similar phrase involving the term “may,” “can,” or “could” is used, it should be understood that the given action, feature, element, component, attribute, etc. is present in at least one embodiment, though is not necessarily present in all embodiments. Terms and phrases used in this document, and variations thereof, unless otherwise expressly stated, should be construed as open-ended rather than limiting. As examples of the foregoing: “and/or” includes any and all combinations of one or more of the associated listed items (e.g., a and/or b means a, b, or a and b); the singular forms “a”, “an” and “the” should be read as meaning “at least one,” “one or more,” or the like; the term “example” is used provide examples of the subject under discussion, not an exhaustive or limiting list thereof; the terms “comprise” and “include” (and other conjugations and other variations thereof) specify the presence of the associated listed items but do not preclude the presence or addition of one or more other items; and if an item is described as “optional,” such description should not be understood to indicate that other items are also not optional.

As used herein, the term “non-transitory computer-readable storage medium” includes a register, a cache memory, a ROM, a semiconductor memory device (such as a D-RAM, S-RAM, or other RAM), a magnetic medium such as a flash memory, a hard disk, a magneto-optical medium, an optical medium such as a CD-ROM, a DVD, or Blu-Ray Disc, or other type of device for non-transitory electronic data storage. The term “non-transitory computer-readable storage medium” does not include a transitory, propagating electromagnetic signal.

### Example Embodiments

Embodiment 1: A method for monitoring heart function to assess for treatment of congestive heart failure and other conditions, the method comprising: processing a plurality of images of a left ventricle of a heart of a subject, the plurality of images corresponding to different points in time in which the heart beats; calculating, using at least one hardware processor of a computing system, multiple displacement values that each correspond to an amount by which a corresponding portion of the endocardial border of the left ventricle of the heart displaces towards a centerline of the left ventricle; and generating and displaying, on a display that is coupled to the computing system, a graphical representation that illustrates the contractility of the left ventricle based on the calculated multiple displacement values.

Embodiment 2: The method of Embodiment 1, further comprising: assessing, based on the calculated multiple displacement values, whether the left ventricle of the subject is suitable for therapeutic treatment.

Embodiment 3: The method of Embodiment 1 or 2, further comprising: determining, based on the calculated multiple displacement values, that the heart is not suitable for therapeutic treatment.

Embodiment 4: The method of any of Embodiments 2 to 3, further comprising: determining, based on the calculated multiple displacement values, that the heart is suitable for therapeutic treatment.

Embodiment 5: The method of any of Embodiments 2 to 4, further comprising: performing the therapeutic treatment that modifies at least one physical property of the left ventricle of the subject.

Embodiment 6: The method of any of Embodiments 2 to 5, wherein the therapeutic treatment includes injecting a hydrogel within the myocardium of the heart of the subject.

Embodiment 7: The method of any of Embodiments 2 to 5, wherein the therapeutic treatment includes performing ventricular reconstruction by deploying at least one anchor to the left ventricle.

Embodiment 8: The method of Embodiment 7, wherein the ventricular reconstruction includes brining opposing walls of the left ventricle into contact with one another by using the at least one anchor.

Embodiment 9: The method of any of Embodiments 2 to 7, wherein a location of where the therapeutic treatment is performed within the heart is based on at least one of the calculated multiple displacement values.

Embodiment 10: The method of any of Embodiments 1 to 9, wherein the plurality of images correspond to a plurality of different planar cuts of the left ventricle.

Embodiment 11: The method of Embodiment 10, wherein the centerline of the left ventricle is at an intersection of at least two of the plurality different planer cuts.

Embodiment 12: The method of any of Embodiments 1 to 11, further comprising: comparing the calculated multiple displacement values to displacement values that are based on a pool of normal subjects.

Embodiment 13: The method of Embodiment 12, wherein the graphical representation includes displaying a percentage value that is based on the comparison.

Embodiment 14: The method of any of Embodiments 1 to 13, further comprising: calculating a plurality of segment displacement values from the multiple displacement values, wherein each of the plurality of segment displacement values corresponds to a different one of a plurality of segments of the left ventricle, wherein at least some of the plurality of segment displacement values are based on two or more of the multiple displacement values that are associated with different areas of the endocardial border.

Embodiment 15: The method of any one of Embodiments 1 to 14, further comprising: calculating a plurality of region displacement values from the multiple displacement values, wherein each of the plurality of region displacement values corresponds to a different one of a plurality of regions of the left ventricle, wherein at least some of the plurality of region displacement values are based on two or more of the multiple displacement values that are associated with different segments of the left ventricle.

Embodiment 16: The method of Embodiment 15, wherein the plurality of regions includes at least a basal region, a mid region, and an apical region.

Embodiment 17: The method of any one of Embodiments 1 to 16, wherein each of the multiple displacement values represents a quantifiable value of contractility of a given segment or region within the left ventricle of the heart of the subject.

Embodiment 18: The method of any one of Embodiments 1 to 17, further comprising: placing a plurality of markers on each of the plurality of images.

Embodiment 19: The method of Embodiment 17, further comprising: deriving, for each of the plurality of images, where the endocardial border of the left ventricle of the heart is located within the image based on the placed markers.

Embodiment 20: The method of any one of Embodiments 1 to 19, wherein different ones of the plurality of images represent different points in time during a heartbeat.

Embodiment 21: The method of Embodiment 20, wherein at least one of the plurality of images represents the left ventricle at end diastole (ED) and at least one of the plurality of images represents the left ventricle at end systole (ES).

Embodiment 22: The method of any one of Embodiments 1 to 21, wherein generating the graphical representation includes aggregating data on movement of the markers between different ones of the plurality of images.

Embodiment 23: The method of any of Embodiments 1-22, wherein the graphical representation includes a graphical indication of the endocardial border of the left ventricle at ED and a graphical indication of the endocardial border of the left ventricle at ES.

Embodiment 24: The method of any of Embodiments 1-23, wherein the graphical representation includes representations for each of the calculated multiple displacement values.

Embodiment 25: The method of Embodiment 24, wherein the representation of the calculated multiple displacement values includes a plurality of displacement vectors that are each based on a corresponding one of calculated multiple displacement values.

Embodiment 26: The method of Embodiment 24, wherein the representation of the calculated multiple displacement values is further based on showing normal contractility, hypokinesia, and/or akinesia of the heart wall.

Embodiment 27: The method of any of Embodiments 1-26, wherein colors and/or shading of the graphical representation are adjusted to indicate normal contractility, hypokinesia, and/or akinesia of the heart wall.

Embodiment 28: The method of any of Embodiments 1-27, wherein the graphical representation includes a bullseye chart with each area of the bullseye chart showing at least one of the calculated multiple displacement values.

Embodiment 29: The method of Embodiment 28, further comprising: comparing results from the bullseye chart to obtained global strain results.

Embodiment 30: The method of Embodiment 29, wherein the global strain results are present in a second bullseye chart.

Embodiment 31: The method of any of Embodiments 1-30, further comprising: plotting a path of a marker as the heart contracts and relaxes as represented over the plurality of images.

Embodiment 32: The method of Embodiment 31, wherein calculation of the multiple displacement values if further based on motion of the marker and/or the plotted path.

Embodiment 33: The method of any of Embodiments 1-32, further comprising: calculating a plurality of vectors that show a direction and an amount of movement of the heart at different points in time during contraction and/or relaxation of the heart.

Embodiment 34: The method of Embodiment 33, wherein the multiple displacement values are calculated as a function of the calculated plurality of vectors.

Embodiment 35: The method of Embodiment 2, further comprising: determining an indication or prediction of success in performing a medical procedure for the therapeutic treatment.

Embodiment 36: The method of Embodiment 35, wherein the medical procedure includes reconstruction of the left ventricle by placing anchors.

Embodiment 37: The method of Embodiment 2, wherein the therapeutic treatment is one of multiple possible heart procedures.

Embodiment 38: The method of Embodiment 37, wherein one of the multiple possible heart procedures involves using anchors for the left ventricle.

Embodiment 39: The method of Embodiment 37, wherein one of the multiple possible heart procedures include injecting a hydrogel into the heart wall of the left ventricle.

Embodiment 40: The method of any one of Embodiments 37-39, further comprising selecting one of the multiple possible heart procedures based on the calculated displacement values.

Embodiment 41: A non-transitory computer readable storage medium storing instructions for use with a computer system, the computer system including at least one hardware processor, the stored instructions comprising instructions that are configured to cause the at least one hardware processor to perform operations comprising the method of any one of Embodiments 1-40.

Embodiment 42: A computer system comprising: a processing system comprising instructions that, when executed by the at least one hardware processor of included with the processing system, are configured to cause the at least one hardware processor to perform operations comprising the method of any one of Embodiments 1-40.

### ADDITIONAL APPLICATIONS OF DESCRIBED SUBJECT MATTER

Although process steps, algorithms or the like, including without limitation with reference to FIG. 15, may be described or claimed in a particular sequential order, such processes may be configured to work in different orders. In other words, any sequence or order of steps that may be explicitly described or claimed in this document does not necessarily indicate a requirement that the steps be performed in that order; rather, the steps of processes described herein may be performed in any order possible. Further, some steps may be performed simultaneously (or in parallel) despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary, and does not imply that the illustrated process is preferred.

Although various embodiments have been shown and described in detail, the claims are not limited to any particular embodiment or example. None of the above description should be read as implying that any particular element, step, range, or function is essential. All structural and functional equivalents to the elements of the above-described embodiments that are known to those of ordinary skill in the art are expressly incorporated herein by reference and are intended to be encompassed. Moreover, it is not necessary for a device or method to address each and every problem sought to be solved by the present invention, for it to be encompassed by the invention. No embodiment, feature, element, component, or step in this document is intended to be dedicated to the public.

1. A computer-implemented method for monitoring heart function for treatment of congestive heart failure and other conditions, the method comprising:
obtaining a plurality of images of a patient's heart or a portion thereof;
obtaining an inward displacement measurement of at least a region or segment of the heart based on the difference in the heart wall position between different images of the plurality of images;
assessing the regional contractility of the heart wall motion based on the inward displacement measurement; and
providing a graphical representation of the regional contractility of the heart wall for determining regions or segments of the heart that are suitable or eligible for therapeutic treatment.

2. The method of claim 1 wherein:
obtaining a plurality of images of a patient's heart or a portion thereof comprises obtaining a series of cuts of the heart; and
wherein obtaining an inward displacement measurement of at least one region of the heart based on the difference between the heart wall position comprises, for each of the cuts of the heart:
determining a distance from a centerline to the end diastole and the centerline to end systole; and
assigning a displacement by subtracting the distance from the centerline to the end diastole and the distance from the centerline to end systole.

3. The method of claim 2 wherein the series of cuts comprises at least a cut along the vertical long axis and the horizontal long axis through the left ventricle.

4. The method of claim 2 further comprising obtaining at least six cuts of the heart.

5. The method of claim 2 wherein the centerline corresponds to the intersection of the series of cuts.

6. The method of claim 1 further comprising analyzing the plurality of images and determining the endocardial border outlining the dimensions of the heart in each of the plurality of images.

7. The method of claim 6 wherein obtaining an inward displacement measurement of at least one region of the heart comprises determining the position of the endocardial border of the heart in diastole and systole.

8. The method of claim 6 further comprising:
placing at least one marker on the endocardial border, and
wherein obtaining an inward displacement measurement of at least a region of the heart based on the difference between the heart wall position in different images of the plurality of images comprises determining movement of the marker between respective images of the plurality of images.

9. The method of claim 8 wherein assessing the regional contractility of various regions of the heart wall motion based on the inward displacement measurement comprises averaging the determined movement of a plurality of markers.

10. The method of claim 8, wherein obtaining an inward displacement measurement of at least a region of the heart based on the difference between the heart wall position in different images of the plurality of images comprises determining movement of the marker relative to the centerline between respective images of the plurality of images.

11. The method of claim 1, wherein at least one of the images of the plurality of images corresponds to the heart at end systole and at least another of the images of the plurality of images to the heart at end diastole, and wherein obtaining an inward displacement measurement of at least a region of the heart based on the difference between the heart wall position in different images of the plurality of images comprises determining the displacement of the region of the heart between end systole and end diastole.

12. The method of claim 1, wherein assessing the regional contractility of various regions of the heart wall motion based on the inward displacement measurement comprises comparing the inward displacement measurement to a pool of normal subjects.

13. The method of claim 1, wherein the graphical representation comprises an indication of the percentage of inward displacement compared to a normal heart.

14. The method of claim 1, wherein the graphical representation is in the form of a bullseye chart.

15. The method of claim 1, further comprising determining areas of the heart that are:
suitable for therapeutic treatment by ventricular reconstruction by means of anchor deployment based on the assessed regional contractility; and/or
suitable for injection of hydrogels within the myocardium based on the assessed regional contractility.

16. A non-transitory computer readable storage medium storing instructions for use with a computer system, the computer system including at least one hardware processor, the stored instructions comprising instructions that are configured to cause the at least one hardware processor to perform operations comprising:
obtaining a plurality of images of a patient's heart or a portion thereof;
obtaining an inward displacement measurement of at least a region or segment of the heart based on the difference in the heart wall position between different images of the plurality of images;
assessing the regional contractility of the heart wall motion based on the inward displacement measurement; and
providing a graphical representation of the regional contractility of the heart wall for determining regions or segments of the heart that are suitable or eligible for therapeutic treatment.

17. A computer system comprising:
a processing system comprising instructions that, when executed by the at least one hardware processor of included with the processing system, are configured to cause the at least one hardware processor to perform operations comprising:
obtaining a plurality of images of a patient's heart or a portion thereof;
obtaining an inward displacement measurement of at least a region or segment of the heart based on the difference in the heart wall position between different images of the plurality of images;
assessing the regional contractility of the heart wall motion based on the inward displacement measurement; and
providing a graphical representation of the regional contractility of the heart wall for determining regions or segments of the heart that are suitable or eligible for therapeutic treatment.

18. The non-transitory computer readable storage medium of claim 16, wherein the operation further comprise:
obtaining a plurality of images of a patient's heart or a portion thereof comprises obtaining a series of cuts of the heart; and
wherein obtaining an inward displacement measurement of at least one region of the heart based on the difference between the heart wall position comprises, for each of the cuts of the heart:
determining a distance from a centerline to the end diastole and the centerline to end systole, and
assigning a displacement by subtracting the distance from the centerline to the end diastole and the distance from the centerline to end systole.
